(19) United States
(12) Patent Application Publication
(10) Pub. No.: US 2024/0416335 A1
(43) Pub. Date: Dec. 19, 2024

(54) MULTICHANNEL PIPETTE AND ELECTROPORATION SYSTEM

(71) Applicants: Life Technologies Holdings PTE Limited, Singapore (SG); Life Technologies Corporation, Carlsbad, CA (US)

(72) Inventors: Wui Khen Kevin LIAW, Singapore (SG); Kuan Moon Boo, Singapore (SG); Huei Steven YEO, Singapore (SG); Wern Yuh FOO, Singapore (SG); Joshua Mathew MEAD, Oceanside, CA (US); Justina Linkai BONG, Singapore (SG); Mikko MAKINEN, Santa Clara, CA (US); Mio Xiu Lu LING, Singapore (SG); Way Xuang LEE, Singapore (SG); Wen Lun CHAN, Singapore (SG); Yunxiang LIU, Singapore (SG)

(21) Appl. No.: 18/739,087

(22) Filed: Jun. 10, 2024

Related U.S. Application Data
(60) Provisional application No. 63/472,208, filed on Jun. 9, 2023.

(51) Int. Cl. B01L 3/02

(52) U.S. Cl. CPC B01L 3/0217; B01L 3/0279; B01L 2300/0645

(57) ABSTRACT

Multichannel pipettes, electroporation systems utilizing the multichannel pipettes and methods for electroporating a cell. The electroporation system includes a multichannel pipette, a pipette tip(s), a pipette docking assembly, and a pulse generator. The pipette docking assembly includes a pipette station, a pipette station guard, and a reservoir.

### CROSS REFERENCE TO RELATED APPLICATION(S)

This application claims the benefit of priority to U.S. Provisional Patent Application No. 63/472,208, filed Jun. 9, 2023, the disclosure of which is considered part of, and incorporated in its entirety by reference in the disclosure of this application.

### BACKGROUND

### Field

The present invention relates generally to devices for fluidic manipulation, and more particularly to a multichannel pipette, an electroporation system utilizing the multichannel pipette and methods for electroporating a cell.

### Background Information

Transfection involves introducing nucleic acids and/or proteins into cells in a nonviral manner. Various chemical or physical methods may be employed to facilitate transfection. Electroporation is a physical transfection method that utilizes an electrical pulse to create temporary pores in cell membranes, through which substances like nucleic acids can pass into cells.

Some electroporation systems include a pipette for holding the target cells and the payload (e.g., nucleic acid and/or proteins to be introduced into the target cells) and an electrical pulse generator for providing an electrical pulse to the target cells. The pipette can be connected to or inserted into a docking station associated with the electrical pulse generator to enable the electrical pulse generated by the electrical pulse generator to reach the target cells.

For example, a pipette electrode in conductive communication with one end of a pipette tip containing liquid including the target cells and payload can interface with a first electrode on the docking station. An opposing end of the pipette tip can be inserted into a buffer solution (e.g., within a reservoir or buffer tube) that is in conductive communication with a second electrode on the docking station, thereby exposing the open end of the pipette tip to the buffer solution. With the pipette and pipetted tip so connected to the docking station, the electrical pulse generator can provide an electrical pulse to the first and second electrodes of the docking station, thereby allowing the electrical pulse to travel through the pipette and pipette tip to reach the target cells.

Conventional electroporation systems include a single channel pipette and are therefore limited in high throughput applications. As such, a disadvantage of conventional electroporation systems is the inability to electroporate multiple different samples at one time which is desirable for high throughput. The ability to process multiple sample at one time vastly decreases processing time, as well as the amount of labor required by technicians in processing high sample numbers.

Existing electroporation systems suffer from a number of shortcomings and there is an ongoing need and desire for improved electroporation systems which can be applied in high throughput applications.

### SUMMARY

Various aspects of the present disclosure extend at least to electroporation systems, components thereof, and/or methods associated therewith.

In one aspect, the present disclosure provides a multichannel pipette (also referred to herein as “pipette”). The pipette includes: a proximal section having a handle; a distal section configured to reversibly engage a plurality of pipette tips; a first actuator disposed in the proximal section; and a second actuator disposed in the proximal section operable to control dispensing function of the pipette In various embodiments, the first actuator is operable to control aspiration and the second actuator is operable to control dispensing.

In various embodiments, the pipette also includes a third actuator disposed in the proximal section configured to cause a pipette tip attached to the distal section of the pipette to disengage when the third actuator is actuated. In some embodiments, the first actuator is configured to transition from a first undepressed position to a second depressed position, wherein the second actuator is configured to transition from a first depressed position to a second undepressed position when the first actuator is transitioned from the first undepressed position to the second depressed position, and wherein transitioning the first actuator from the first undepressed position to the second depressed position causes an aspiration function and transitioning of the second actuator from the second undepressed position to the first depressed position causes a dispensing function.

In various embodiments, the pipette includes a plurality of gripper mechanisms disposed in the distal section, each gripper mechanism being configured to reversibly grip a plunger disposed within a lumen of a pipette tip, the plunger having an engagement section for engaging the gripper mechanism and a lumen section disposed within a lumen of a pipette tip.

In some embodiments, the pipette includes a lock button disposed in the proximal section operably connected to a lock mechanism configured to control locking of the plurality of gripper mechanisms. Depressing the lock button causes the lock mechanism to transition from a first unlocked configuration to a second locked configuration. Additionally, when the lock mechanism is in the first configuration the plurality of gripper mechanisms are open and configured to receive the engagement section of the plunger, and when the lock mechanism is in the second configuration the plurality of gripper mechanisms are closed and configured to be in grasping engagement with the engagement section of the plunger.

In another aspect, the present disclosure provides an electroporation system that includes a pipette of the disclosure, a pipette tip, a pipette docking assembly, and a pulse generator. In some embodiments, the pipette docking assembly includes a pipette station, a pipette station guard, and a reservoir.

In still another aspect, the present disclosure provides a method for transfecting a cell with a payload. The method includes providing an electroporation system of the disclosure, providing the cell, providing the payload, introducing the cell and the payload into a pipette tip, and electroporating the cell by operating the electroporation system. In some embodiments, the cell is a mammalian cell In some embodiments, the payload includes a nucleic acid, a protein, or a combination thereof.

### BRIEF DESCRIPTION OF THE DRAWINGS

For a fuller understanding of the nature and advantages of the present invention, reference should be made to the following detailed description taken in connection with the accompanying drawings. It is appreciated that these drawings depict only typical embodiments of the invention and are therefore not to be considered limiting of its scope. The disclosure will be described and explained with additional specificity and detail through the use of the accompanying drawings.

FIG. 1 is a schematic diagram illustrating example components of an electroporation system of the disclosure, as well as components thereof in one embodiment of the disclosure.

FIG. 2 is a front, right, elevated perspective view of one embodiment of a multichannel pipette of the disclosure.

FIG. 3 right side view of the multichannel pipette depicted in FIG. 2.

FIG. 4 left side view of the multichannel pipette depicted in FIG. 2.

FIG. 5 back side view of the multichannel pipette depicted in FIG. 2.

FIG. 6 front side view of the multichannel pipette depicted in FIG. 2.

FIG. 7 top side view of the multichannel pipette depicted in FIG. 2.

FIG. 8 bottom side view of the multichannel pipette depicted in FIG. 2.

FIG. 9 is a front, right, elevated perspective view of one embodiment of a multichannel pipette of the disclosure, wherein the pipette has pipette tips attached to the distal section of the pipette.

FIG. 10 right side view of the multichannel pipette depicted in FIG. 9.

FIG. 11 left side view of the multichannel pipette depicted in FIG. 9.

FIG. 12 back side view of the multichannel pipette depicted in FIG. 9.

FIG. 13 front side view of the multichannel pipette depicted in FIG. 9.

FIG. 14 top side view of the multichannel pipette depicted in FIG. 9.

FIG. 15 bottom side view of the multichannel pipette depicted in FIG. 9.

FIGS. 16A-16B are schematic diagrams illustrating aspects of example pipette tips (e.g., consumable pipette tips) for use with the electroporation system in embodiments of the disclosure. FIG. 16A is a schematic diagram illustrating aspects of an example pipette tip in one embodiment of the disclosure. FIG. 16B is a schematic diagram illustrating aspects of an example pipette tip in one embodiment of the disclosure.

FIGS. 17A-17B are schematic diagrams showing exploded views of the pipette tips depicted in FIGS. 16A-16B. FIG. 17A is an exploded view of the pipette tip depicted in FIG. 16A. FIG. 17B is an exploded view of the pipette tip depicted in FIG. 16B.

FIGS. 18A-18B are schematic diagrams illustrating aspects of plungers of pipette tips in embodiments of the disclosure. FIG. 18A is an exploded view of an example plunger. FIG. 18B is a schematic diagram of the plunger depicted in FIG. 18A as assembled.

FIGS. 19A-19B are schematic diagrams illustrating aspects of a pipette tip in embodiments of the disclosure. FIG. 19A is a cross sectional view of a pipette tip in one embodiment of the disclosure. FIG. 19B is an expanded cross sectional view of the distal portion of the pipette tip depicted in FIG. 19A.

FIG. 20 a schematic diagram illustrating aspects of a plunger of a pipette tip in embodiments of the disclosure.

FIGS. 21A-21B are schematic diagrams illustrating a pipette tip in one embodiment of the disclosure. FIG. 21A is a schematic diagram of a pipette tip of the disclosure FIG. 21B is an expanded view of the distal portion of the pipette tip depicted in FIG. 21A.

FIG. 22 is a perspective view of a pipette tip for use with the multichannel pipette in embodiments of the disclosure.

FIG. 23 is an elevated perspective view of a multichannel pipette in one embodiment of the disclosure.

FIG. 24 is a cross sectional side view of the pipette of FIG. 23.

FIG. 25 illustrates functioning of internal mechanisms of the pipette of FIG. 23 when the second actuator is moved to the first depressed position to dispense fluid.

FIG. 26 illustrates functioning of internal mechanisms of the pipette of FIG. 23 when the first actuator is moved to the second depressed position to aspirate fluid.

FIG. 27 illustrates the positioning of internal components of the pipette when the second actuator is in the first depressed position after a dispensing function is performed or when the pipette is configured to depress the lock button to lock and secure plungers of attached pipette tips.

FIG. 28 illustrates internal components of the pipette operably connecting the lock button with the gripping sleeves via the push plate.

FIG. 29 illustrates the positioning of internal components of the pipette once the lock button has been depressed and the second actuator is fully depressed. The plungers of individual pipette tips are shown gripped and locked via individual gripper mechanisms.

FIG. 30 is an expanded view of the rectangular region bound by dashed line shown in FIG. 28 showing a plunger of a pipette tip locked and grasped by the gripper jaw of the gripper mechanism. The gripping sleeve is moved distally with respect to the distal tip of the gripping sleeve to close the gripper jaw and lock the plunger of the pipette tip with the gripper mechanism.

FIG. 31 is an elevated perspective view of a multichannel pipette in one embodiment of the disclosure.

FIG. 32 illustrates functioning of internal mechanisms of the pipette of FIG. 31 when the second actuator is moved to the first depressed position to dispense fluid.

FIG. 33 illustrates functioning of internal mechanisms of the pipette of FIG. 31 when the first actuator is moved to the second depressed position to aspirate fluid.

FIGS. 34A-34B illustrate the positioning of internal components of the pipette. FIG. 34A illustrates the positioning of internal components of the pipette when the second actuator is in the first depressed position after a dispensing function is performed or when the pipette is configured to depress the lock button to lock and secure plungers of attached pipette tips. FIG. 34B illustrates the positioning of internal components of the pipette when the second actuator is in the first depressed position after a dispensing function is performed or when the pipette is configured to depress the lock button to lock and secure plungers of attached pipette tips.

FIG. 35 illustrates the positioning of internal components of the pipette once the lock button has been depressed and the second actuator is fully depressed. The plungers of individual pipette tips are shown gripped and locked via individual gripper mechanisms.

FIG. 36 is an expanded view of the rectangular region bound by dashed line shown in FIG. 35 showing a plunger of a pipette tip locked and grasped by the gripper jaw of the gripper mechanism. The gripping sleeve is moved distally with respect to the distal tip of the gripper jaw to close the gripper jaw to grasp and lock the plunger of the pipette tip with the gripper mechanism.

FIG. 37 is a schematic diagram illustrating a gripper mechanism in an open configuration such that an engagement section of a pipette tip plunger can be received by the gripper jaw in embodiments of the disclosure.

FIG. 38 is a schematic diagram illustrating the gripper mechanism of FIG. 37 in an open configuration such that an engagement section of a pipette tip plunger can be received by the gripper jaw in embodiments of the disclosure.

FIG. 39 is a schematic diagram illustrating the gripper mechanism of FIG. 37 in an open configuration such that an engagement section of a pipette tip plunger can be received by the gripper jaw in embodiments of the disclosure.

FIG. 40 is a schematic diagram illustrating a gripper mechanism in a closed configuration such that an engagement section of a pipette tip plunger can be grasped by the gripper jaw in embodiments of the disclosure.

FIG. 41 is a schematic diagram illustrating the gripper mechanism of FIG. 40 in a closed configuration such that an engagement section of a pipette tip plunger can be grasped by the gripper jaw in embodiments of the disclosure.

FIG. 42 is a schematic diagram illustrating the placement and interaction between magnets disposed on the outer housing of the multichannel pipette and the internally disposed linkage bar in embodiments of the disclosure.

FIG. 43 is a schematic diagram illustrating the placement and interaction between magnets disposed on the outer housing of the multichannel pipette and the internally disposed linkage bar in embodiments of the disclosure.

FIG. 44 is a schematic diagram illustrating the placement and interaction between magnets disposed on the outer housing of the multichannel pipette and the internally disposed linkage bar in embodiments of the disclosure.

FIG. 45 is a schematic diagram illustrating internal mechanisms and functioning of the pipette shown in FIG. 31 used to displace and remove attached pipette tips in embodiments of the disclosure.

FIG. 46 is a schematic diagram illustrating the linkage bar when the second actuator is in the first depressed position and false trigger prevention of the pipette shown in FIG. 31 in embodiments of the disclosure.

FIG. 47 is a schematic diagram illustrating a step of pipette tip disengagement after false trigger travel of the pipette shown in FIG. 31 in embodiments of the disclosure.

FIG. 48 is a schematic diagram illustrating a step of pipette tip disengagement to disengage and release the attachment interface of a pipette tip simultaneously with plunger disengagement and release of the pipette shown in FIG. 31 in embodiments of the disclosure.

FIG. 49 is a schematic diagram illustrating functionality of the tip ejection sleeve of the pipette shown in FIG. 31 in embodiments of the disclosure.

FIGS. 50A-50B are schematic diagrams illustrating internal components of the pipette utilized in force reduction during disengagement of a pipette tip. FIG. 50A illustrates internal components of the pipette utilized in force reduction including gears. FIG. 50B illustrates an individual gear. Force reduction is achieved using a lever configuration between fulcrum and load.

FIG. 51 is a schematic diagram illustrating internal mechanisms and functioning of the pipette shown in FIG. 31 used to displace and remove attached pipette tips in embodiments of the disclosure.

FIG. 52 is a schematic diagram illustrating the linkage bar when the second actuator is in the first depressed position and false trigger prevention of the pipette shown in FIG. 31 in embodiments of the disclosure.

FIG. 53 is a schematic diagram illustrating partial tip ejection after the third actuator is moved to a partially depressed position and the linkage bar is moved distally but pipette tip plungers remain engaged until the third actuator is fully depressed of the pipette shown in FIG. 31 in embodiments of the disclosure.

FIG. 54 is a schematic diagram illustrating internal mechanisms and functioning of the pipette shown in FIG. 23 used to displace and remove attached pipette tips in embodiments of the disclosure.

FIG. 55 is a schematic diagram illustrating internal mechanisms and functioning of the pipette shown in FIG. 23 used to displace and remove attached pipette tips in embodiments of the disclosure.

FIG. 56 is a schematic diagram illustrating internal mechanisms and functioning of the pipette shown in FIG. 23 used to displace and remove attached pipette tips in embodiments of the disclosure.

FIG. 57 is a schematic diagram illustrating internal mechanisms and functioning of the pipette shown in FIG. 23 used to displace and remove attached pipette tips in embodiments of the disclosure.

FIG. 58 is a schematic diagram illustrating internal mechanisms and functioning of the pipette shown in FIG. 23 used to displace and remove attached pipette tips in embodiments of the disclosure.

FIG. 59 is a diagram illustrating an electroporation system in one embodiment of the disclosure.

FIG. 60 is an elevated front perspective view of the pipette docking assembly shown in FIG. 59 without the pipette and reservoir being docked in embodiments of the disclosure.

FIG. 61 is an elevated back perspective view of the pipette docking assembly shown in FIG. 59 without the pipette and reservoir being docked in embodiments of the disclosure.

FIG. 62 is a schematic diagram illustrating components of the reservoir shown in FIG. 59 in embodiments of the disclosure.

FIG. 63 is a schematic diagram illustrating aspects of a pipette station guard in an embodiment of the disclosure.

FIG. 64 is a schematic diagram illustrating connection of the pipette station guard of FIG. 63 to a pipette docking station.

FIG. 65 is a schematic diagram illustrating connection of the pipette station guard of FIG. 63 to a pipette docking station.

FIG. 66 is a schematic diagram illustrating an alternative configuration of gears for the gear mechanism of the pipette shown in FIG. 32 in embodiments of the disclosure.

FIG. 67 is an elevated perspective view of a multichannel pipette in one embodiment of the disclosure.

FIG. 68 is an elevated perspective view of a multichannel pipette of the disclosure engaging pipette tips.

FIG. 69 illustrates functioning of internal mechanisms of the pipette of FIG. 67 when the lock button is depressed.

FIG. 70 illustrates functioning of internal mechanisms of the pipette of FIG. 67 when the first actuator is moved to the second depressed position to aspirate fluid.

FIG. 71 illustrates the positioning of internal components of the pipette of FIG. 67 before the eject button is depressed.

FIG. 72 illustrates internal components of the pipette of FIG. 67 operably connected to pipette tips with the lock button depressed and with plungers grasped by gripping jaws.

FIG. 73 illustrates gripper jaws of the pipette of FIG. 67 in the closed position.

FIG. 74 illustrates functioning of internal mechanisms of the pipette of FIG. 67 when the first actuator is moved to the second depressed position after which tips may be loaded on one embodiment of the disclosure.

FIG. 75 illustrates gripper jaws of the pipette shown in FIG. 74 when the first actuator is moved to the second depressed position.

FIG. 76 illustrates tips loaded on the pipette shown in FIG. 67.

FIG. 77 illustrates aspects of a gripper mechanism of the pipette shown in FIG. 67.

FIG. 78 is a cross sectional view illustrating aspects of a gripper mechanism of the pipette shown in FIG. 67.

FIG. 79 illustrates aspects of a gripper mechanism of the pipette shown in FIG. 67.

FIG. 80 is a cross sectional view illustrating aspects of a gripper mechanism of the pipette shown in FIG. 67.

FIG. 81 illustrates aspects of a push plate assembly of the pipette shown in FIG. 67.

FIG. 82 illustrates functioning of internal mechanisms of the pipette of FIG. 67 when the lock button is deactivated.

FIG. 83 illustrates aspects of a gripper mechanism of the pipette shown in FIG. 67 with the gripper jaws being open.

FIG. 84 illustrates functioning of internal mechanisms of the pipette of FIG. 67 when the lock button is activated, e.g., depressed.

FIG. 85 illustrates aspects of a gripper mechanism of the pipette shown in FIG. 67 with the gripper jaws being closed.

FIG. 86 illustrates functioning of internal mechanisms of the pipette of FIG. 67 when the lock button is activated, e.g., depressed.

FIG. 87 illustrates functioning of internal mechanisms of the pipette of FIG. 67 when the first actuator is moved to the depressed position to aspirate fluid.

FIG. 88 illustrates functioning of internal mechanisms of the pipette of FIG. 67 when the first actuator is moved to the depressed position to dispense fluid.

FIG. 89 illustrates positioning of internal mechanisms of the pipette of FIG. 67 when the first actuator is moved to the depressed position to aspirate fluid.

FIG. 90 illustrates the positioning of internal components of the pipette of FIG. 67 before the eject button is depressed.

FIG. 91 illustrates aspects of a push plate assembly of the pipette shown in FIG. 67.

FIG. 92 illustrates internal aspects of a pipette station top assembly with anode a cathode modules for electroporation.

FIG. 93 illustrates internal aspects of a pipette station top assembly with anode a cathode modules for electroporation.

FIG. 94 illustrates internal aspects of a pipette station bottom assembly.

FIG. 95 illustrates internal aspects of a pipette station bottom assembly.

FIG. 96 illustrates aspects of a cathode module of a pipette station top assembly.

FIG. 97 illustrates aspects of a cathode module of a pipette station top assembly.

FIG. 98 illustrates aspects of an anode module of a pipette station top assembly.

FIG. 99 illustrates aspects of an anode module of a pipette station top assembly.

FIG. 100 illustrates example components of an electroporation system of the disclosure, in some embodiments.

FIG. 101 is a front, right perspective view of one embodiment of the docking assembly with a pipette docked.

FIG. 102 is a front, right, perspective view of a docking assembly.

FIG. 103 is a front, right, perspective view of a pipette station.

FIG. 104 is a front elevation view of the pipette station of FIG. 103.

FIG. 105 is a rear elevation view of the pipette station of FIG. 103.

FIG. 106 is a right side elevation view of the pipette station of FIG. 103.

FIG. 107 is a left side elevation view of the pipette station of FIG. 103.

FIG. 108 is a top plan view of the pipette station of FIG. 103.

FIG. 109 is a bottom plan view of the pipette station of FIG. 103.

FIG. 110 is a front, right, perspective view of a station guard.

FIG. 111 is a front elevation view of the station guard of FIG. 110.

FIG. 112 is a rear elevation view of the station guard of FIG. 110.

FIG. 113 is a right side elevation view of the station guard of FIG. 110.

FIG. 114 is a left side elevation view of the station guard of FIG. 110.

FIG. 115 is a top plan view of the station guard of FIG. 110.

FIG. 116 is a bottom plan view of the station guard of FIG. 110.

FIG. 117 is a front, right, perspective view of a reservoir.

FIG. 118 is a front elevation view of the reservoir of FIG. 117.

FIG. 119 is a rear elevation view of the reservoir of FIG. 117.

FIG. 120 is a right side elevation view of the reservoir of FIG. 117.

FIG. 121 is a left side elevation view of the reservoir of FIG. 117.

FIG. 122 is a top plan view of the reservoir of FIG. 117.

FIG. 123 is a bottom plan view of the reservoir of FIG. 117.

FIG. 124 is a front, right, perspective view of a multichannel pipette.

FIG. 125 is a front elevation view of the multichannel pipette of FIG. 124.

FIG. 126 is a rear elevation view of the multichannel pipette of FIG. 124.

FIG. 127 is a right side elevation view of the multichannel pipette of FIG. 124.

FIG. 128 is a left side elevation view of the multichannel pipette of FIG. 124.

FIG. 129 is a top plan view of the multichannel pipette of FIG. 124.

FIG. 130 is a bottom plan view of the multichannel pipette of FIG. 124.

FIG. 131 is a front, right, perspective view of a multichannel pipette having pipette tips attached.

FIG. 132 is a front elevation view of the multichannel pipette of FIG. 131.

FIG. 133 is a rear elevation view of the multichannel pipette of FIG. 131.

FIG. 134 is a right side elevation view of the multichannel pipette of FIG. 131.

FIG. 135 is a left side elevation view of the multichannel pipette of FIG. 131.

FIG. 136 is a top plan view of the multichannel pipette of FIG. 131.

FIG. 137 is a bottom plan view of the multichannel pipette of FIG. 131.

FIG. 138 is a front, right, perspective view of a pipette tip plunger in one embodiment of the disclosure.

FIG. 139 is a front elevation view of the pipette tip plunger of FIG. 138.

### DETAILED DESCRIPTION

Implementations of the present disclosure extend at least to pipettes (e.g., multichannel pipettes) used for electroporation, as well as electroporation systems and/or components thereof which utilize such pipettes. The disclosed aspects and embodiments may be implemented to address various shortcomings associated with at least some conventional pipettes and electroporation systems and/or techniques. The following discussion outlines some example improvements and/or practical applications that may be provided by the disclosed embodiments. It will be appreciated, however, that the following are examples only and that the embodiments described herein are in no way limited to the example improvements discussed herein.

Some implementations of the present disclosure provide pipettes that allow simultaneous processing of multiple samples. The unique design of the multichannel pipettes described herein reduces muscular stress and/or fatigue of a user by reducing the forces involved with manually performing pipetting functions on a device that utilizes multiple pipette tips to process multiple samples simultaneously. Additionally, the pipettes of the present disclosure are designed to utilize pipette tips in which a clip-on connection between the pipette tips and pipettes is achieved. Use of clip-on connections along with a reduction in the forces that contribute to muscular stress and/or fatigue during operation of the pipettes makes the pipettes described herein, as well as systems thereof, ideal for high throughput applications.

Before describing various embodiments of the present disclosure in detail, it is to be understood that this disclosure is not limited to the parameters of the particularly exemplified systems, methods, apparatus, products, processes, consumables, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

Furthermore, it is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about,” as that term is defined herein. Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

The term “comprising” which is synonymous with “including,” “containing.” “having” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, reference to a “port” includes one, two, or more ports.

As used in the specification and appended claims, directional terms, such as “top,” “bottom,” “left,” “right,” “up,” “down,” “upper,” “lower,” “inner,” “outer,” “internal,” “external,” “interior,” “exterior,” “proximal,” “distal” and the like are used herein solely to indicate relative directions and are not otherwise intended to limit the scope of the disclosure or claims.

Where possible, like numbering of elements have been used in various figures. Furthermore, alternative configurations of a particular element may each include separate letters appended to the element number. Accordingly, an appended letter can be used to designate an alternative design, structure, function, implementation, and/or embodiment of an element or feature without an appended letter. For instance, an element “80” may be embodied in an alternative configuration and designated “80a.” Similarly, multiple instances of an element and or sub-elements of a parent element may each include separate letters appended to the element number. In each case, the element label may be used without an appended letter to generally refer to all instances of the element or any one of the alternative elements. Element labels including an appended letter can be used to refer to a specific instance of the element or to distinguish or draw attention to multiple uses of the element.

Various aspects of the present devices, systems, and methods may be illustrated with reference to one or more exemplary embodiments. As used herein, the term “embodiment” means “serving as an example, instance, or illustration,” and should not necessarily be construed as preferred or advantageous over other embodiments disclosed herein.

Various aspects of the present devices and systems may be illustrated by describing components that are coupled, attached, and/or joined together. As used herein, the terms “coupled”, “attached”, “connected” and/or “joined” are used to indicate either a direct connection between two components or, where appropriate, an indirect connection to one another through intervening or intermediate components. In contrast, when a component is referred to as being “directly coupled”, “directly attached”, “directly connected” and/or “directly joined” to another component, there are no intervening elements present.

Unless defined otherwise, all technical and scientific terms used herein have the same meaning as commonly understood by one of ordinary skill in the art to which the present disclosure pertains. Although a number of methods and materials similar or equivalent to those described herein can be used in the practice of the present disclosure, the preferred materials and methods are described herein.

### Electroporation System

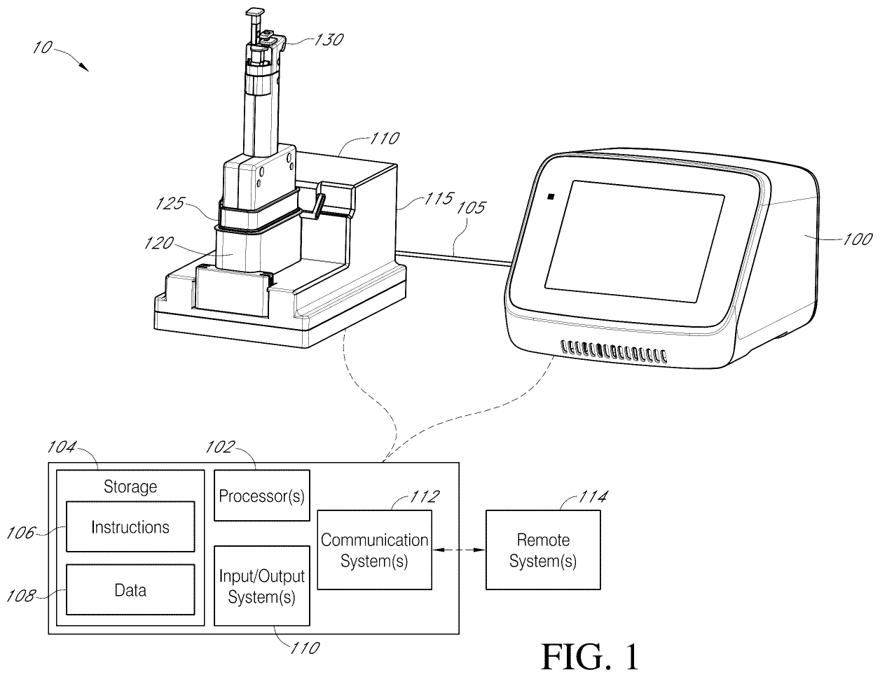

FIG. 1 illustrates various components of an electroporation system 10 that may be used to implement one or more disclosed embodiments. For example, the electroporation system 10 of FIG. 1 may be configured to facilitate cellular transfection by applying an electrical current to target cells to introduce a payload into the target cells. Although FIG. 1 illustrates an electroporation system 10 as including particular components, one will appreciate, in view of the present disclosure, that an electroporation system 10 may include any number of additional and/or alternative components. Furthermore, one will appreciate, in view of the present disclosure, that the principles disclosed herein are not limited to the particular form and/or features of the electroporation system 10 shown in FIG. 1.

FIG. 1 illustrates an electroporation system 10 that includes processor(s) 102, storage 104, input/output system(s) 110 (I/O system(s) 110), and communication system(s) 112. The processor(s) 102 may include one or more sets of electronic circuitries that include any number of logic units, registers, and/or control units to facilitate the execution of computer-readable instructions (e.g., instructions that form a computer program). Such computer-readable instructions may be stored within storage 104. The storage 104 may include physical system memory and may be volatile, non-volatile, or some combination thereof. Furthermore, storage 104 may include local storage, remote storage (e.g., accessible via communication system(s) 112 or otherwise), or some combination thereof. Additional details related to processors (e g., processor(s) 102), computer storage media (e.g., storage 104), and other computer components will be provided hereinafter.

The processor(s) 102 may be configured to execute instructions 106 stored within storage 104 to perform certain actions and/or commands (e.g., voltage/current control, user interface presentation, receiving user input, component detection, etc.). The actions may rely at least in part on data 108 stored on storage 104 in a volatile or non-volatile manner.

In some instances, the actions may rely at least in part on communication system(s) 112 for receiving data from remote system(s) 114, which may include, for example, computing devices, sensors, and/or others. The communications system(s) 112 may include any combination of software or hardware components that are operable to facilitate communication between on-system components/devices and/or with off-system components/devices. For example, the communications system(s) 112 may include ports, buses, or other physical connection apparatuses for communicating with other devices/components. Additionally, or alternatively, the communications system(s) 112 may include systems/components operable to communicate wirelessly with external systems and/or devices through any suitable communication channel(s), such as, by way of non-limiting example, Bluetooth, ultra-wideband, WLAN, infrared communication, and/or others.

Furthermore, FIG. 1 illustrates that an electroporation system 10 may include or be in communication with I/O system(s) 110. I/O system(s) 110 may include any type of input or output device such as, by way of non-limiting example, a display, a touch screen, a mouse, a keyboard or button interface, a controller, and/or others, without limitation. For instance, FIG. 1 illustrates that the electroporation system 10 includes a user interface element implemented in the form of a graphical touch-screen user interface on a pulse generator 100. The user interface element is configured to display information related to operation of the electroporation system 10 and/or receive user input for facilitating control of the electroporation system 10 (e.g., to select parameters for, initiate, monitor, optimize and/or end electroporation processes).

In various embodiments, the system includes one or more machine learning modules. Such modules may be utilized to optimize parameters of one or more aspects of the system described herein, for example to generate and/or optimize protocols, data storage, event/alarm chronicling, batch control, functional operation between instruments and the like.

In certain aspects the present disclosure provides a system for electroporation that includes a computing device (e.g., remote server or otherwise in electronic communication with the system) comprising one or more processors, and a memory, wherein the memory stores instructions that, when executed by the one or more processors, cause the computing device to perform any of the methods as disclosed herein. For example, the memory can store one or more weights associated with one or more trained machine learning models including, for example, one or more trained neural networks, as disclosed herein. The term “weights” as used herein in reference to a neural network, refers to all parameter values and network structure definitions necessary to propagate input data through the neural network in order to obtain an output value.

The system embodiments disclosed herein may achieve improved performance relative to conventional approaches. As such, in embodiments, this invention utilizes a machine learning model, for example a neural network that, combined with an integrated, fast computational architecture, allows for operational improvement of instruments, individually or as an instrument set and/or system.

Various methods and systems of the embodiments disclosed herein may improve upon conventional approaches to achieve the technical advantages of higher throughput, more exact algorithms, and faster, more robust processing by making use of a machine learning model, for example a trained neural network that allows for real-time processing of data, and displaying of representations of bioprocessing data. Such technical advantages are not achievable by routine and conventional approaches, and all users of systems including such embodiments may benefit from these advantages, for example, by assisting the user in the performance of a technical task, such as real-time high-throughput processing, by means of a guided human-machine interaction process. The technical features of the embodiments disclosed herein are thus decidedly unconventional in the field of fill and finish instruments, as are the combinations of the features of the embodiments disclosed herein. The present disclosure thus introduces functionality that neither a conventional computing device, nor a human, could perform. As used herein, the term ‘weights,’ in reference to a neural network, refers to all parameter values and network structure definitions necessary to propagate input data through the neural network to obtain an output value.

The electroporation system 10 includes various physical components that are usable to facilitate electroporation operations. For example, FIGS. 1 and 100 illustrate that the electroporation system 10 includes a pulse generator 100 that is configured to supply electrical pulses to other components of the electroporation system 10. The pulse generator 100 may supply the electrical pulses via cable(s) 105, which may selectively connect the pulse generator 100 to one or more other components of the electroporation system 10. For example, the cable(s) 105 may connect the pulse generator 100 to a pipette docking assembly 110 to supply electrical current to target cells residing within a pipette tip of a multichannel pipette 130 connected to the pipette docking assembly 110. The pipette docking assembly 110 may include a pipette docking station 115 (to which the cable(s) 105 may connect), a pipette station guard 120, and a reservoir 125 configured to hold an electrolytic buffer and which receives the distal section of the pipette 130, as well as pipette tips attached thereto. Additional aspects of components of the electroporation system 10 will be described in more detail hereinbelow.

### Pipette Tips

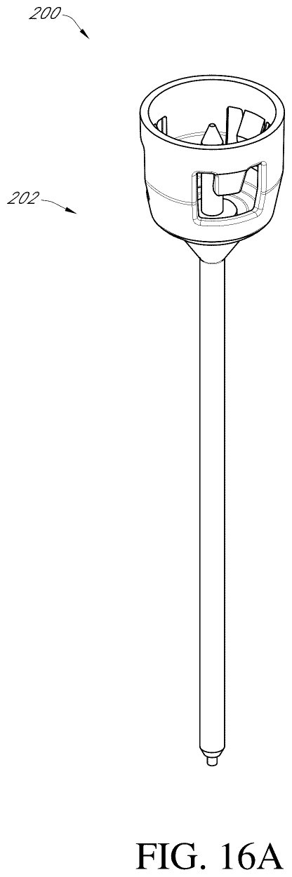

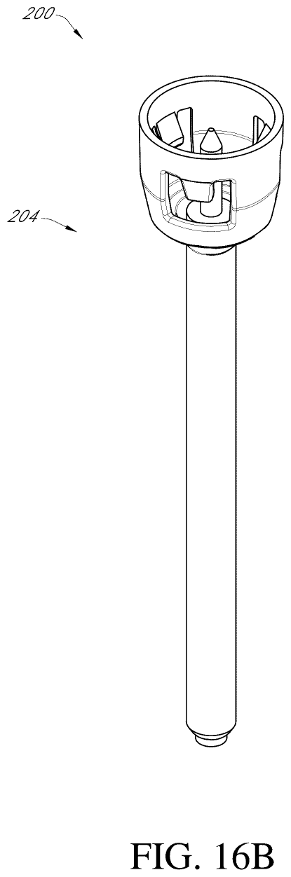

FIGS. 16 through 21 illustrate aspects of pipette tip(s) 200 for use with the multichannel pipette 130 and electroporation system 10 of the disclosure. Such pipette tips may be connected to a pipette (e.g., pipette 130) and may hold cells and a payload to facilitate electroporation. FIG. 16 depicts an example of a 10 μL pipette tip 202 and a 100 μL pipette tip 204. Although only 10 μL and 100 μL sizes are shown in FIG. 16, other sizes are within the scope of the present disclosure.

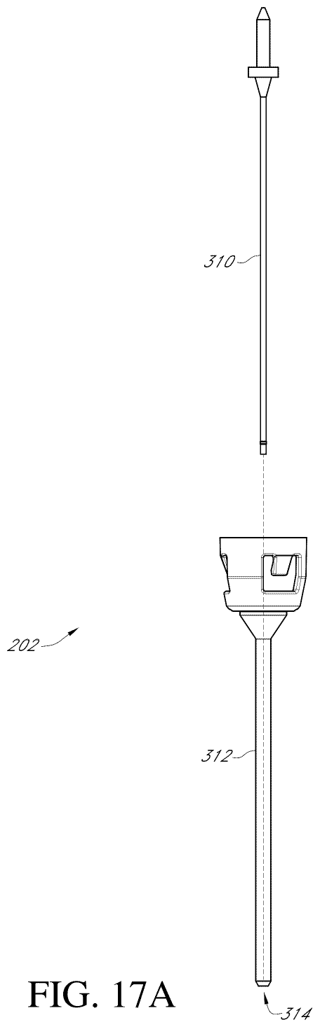

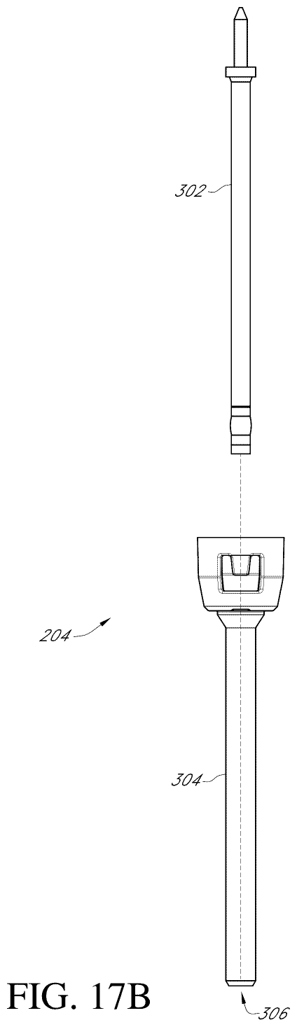

FIG. 17 shows that the 100 μL pipette tip 204 may include a plunger 302 that is configured to be at least partially disposed within a lumen 304 (FIG. 16 shows the plunger 302 fully inserted into the lumen 304). The plunger 302 is configured to translate along the lumen 304 to facilitate pipetting functions (e.g., aspirating and/or dispensing). For instance, the open end 306 of the lumen 304 may be positioned within a reservoir containing cells and a payload (e.g., nucleic acid, protein(s), etc.), and the plunger 302 may be drawn away from the open end 306 of the lumen 304 to draw the cells and the payload into the lumen 304. The 100 μL pipette tip 204 may then be connected to other components of an electroporation system (e.g., the pipette docking assembly 110 of FIG. 1) to electroporate the cells to introduce the payload into the cells.

At least a portion of the plunger 302 may include a conductive material to enable an electrical pulse to reach and/or travel through the contents of the lumen 304. For example, the plunger 302 may be coated with, formed from, or otherwise include a gold (e.g., gold plating), diamond-like carbon, conductive plastic, and/or any other conductive medical-grade materials (e.g., materials that are inert to mammalian cells).

FIG. 17 also shows that the 10 μL pipette tip 202 may include a plunger 310 and lumen 312 (with an open end 314), similar to the plunger 302 and lumen 304 of the 100 μL pipette tip 204.

For conventional pipette tips, a seal is created by the plunger and the lumen by a metal ring on the plunger that interfaces with the inner surface of the lumen. The amount of frictional force exhibited between the metal ring and the lumen can affect the push/pull force required to operate the pipette. The amount of frictional force exhibited between the metal ring and the lumen can be affected by the amount of interference between the metal ring and the lumen. By way of illustrative example, for a 10 μL tip, an interference within a range of 0 to 30 μm can give rise to a push/pull force within a range of 0 to 6 N to operate the pipette. For a larger tip, such as a 10 μL tip, an interference within a range of 0 to 10 μm can give rise to a push/pull force within a range of 0 to 6 N to operate the pipette. It can be difficult to consistently and reliably achieve an interference within the range of 0 to 10 μm in production, which can give rise to pipette tips (particularly larger pipette tips) that have an excessive interference between the metal ring and the lumen, leading to an excessive push/pull force necessary to operate the pipette (e.g., exceeding 6 N).

Accordingly, at least some pipette tips 200 of the present disclosure may implement an alternative sealing component for creating a seal between the lumen and the plunger. This can be particularly beneficial for pipette tips of larger sizes (e.g., 100 μL pipette tips).

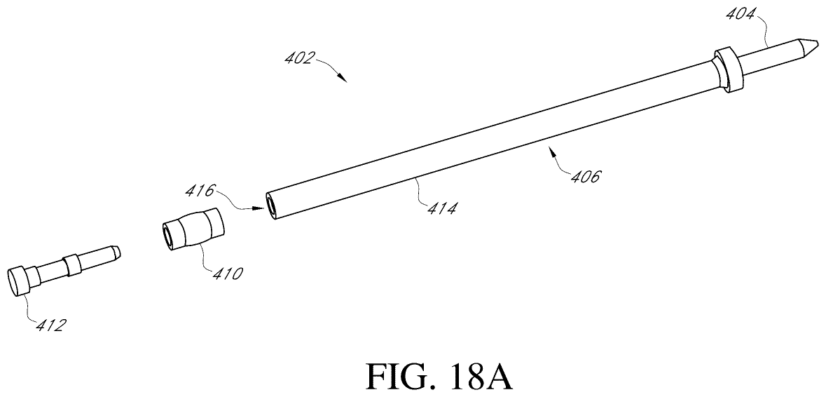

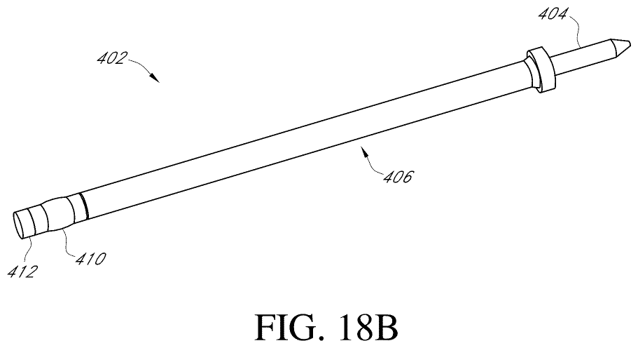

FIG. 18 illustrates an example plunger 402 of a 100 μL pipette tip (in both exploded and assembled configurations) In the example of FIG. 18, the plunger 402 includes an engagement section 404 and a lumen section 406. The engagement section 404 is configured to engage with portions of a gripper mechanism (e.g., a gripper jaw) of a multichannel pipette 130, as will be described in more detail hereinafter. The lumen section 406 is configured to be positioned within and translate along the lumen of a pipette tip.

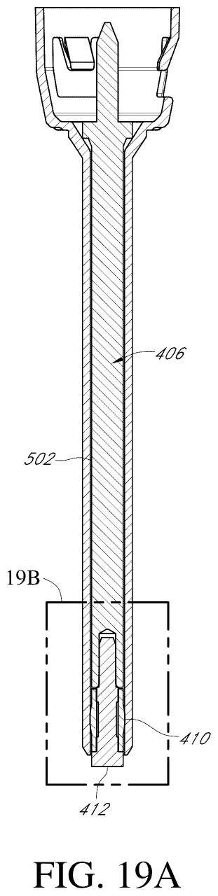

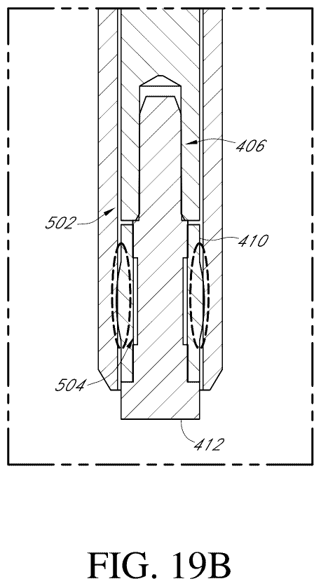

As shown in FIG. 18, the lumen section includes a sealing component 410, which creates a seal between the plunger 402 and the lumen within which the plunger is positioned. FIG. 19 shows a section view of the lumen section 406 of the plunger 402 positioned within a lumen 502. FIG. 19 depicts an interference area between the sealing component 410 and the lumen 502. Advantageously, the interference area does not extend along the entire length of the lumen section 406 of the plunger 402 that is positioned within the lumen 502, thereby facilitating reduced frictional force between the plunger 402 and the lumen 502.

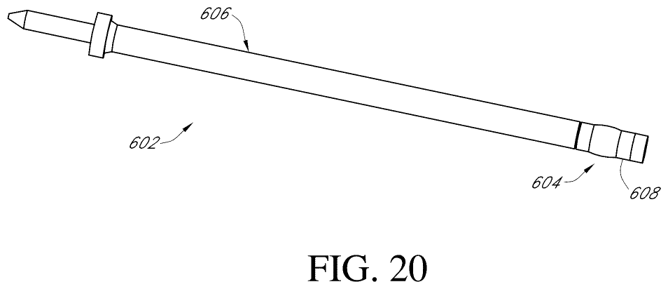

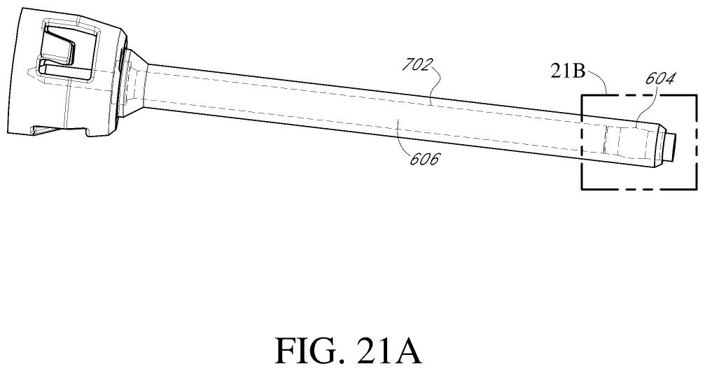

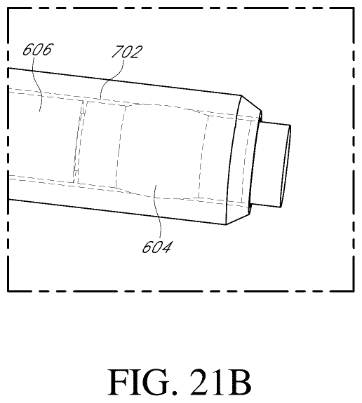

In the example of FIGS. 18 and 19, the sealing component 410 is implemented as a polymer sleeve (other forms are possible, such as an O-ring design as shown in FIGS. 20 and 21). The sealing component 410 may include various types of materials, such as polytetrafluoroethylene (PTFE), other Teflon materials, and/or other pliable and biocompatible materials.

The sealing component 410 may be affixed to the lumen section 406 of the plunger 402 in various ways. In the example of FIGS. 18 and 19, the lumen section includes a front pin 412 and a shaft section 414. The front pin 412 is configured to connect to the shaft section 414, such as by insertion of a portion of the front pin 412 into a retention hole 416 of the shaft section 414. The front pin 412 may further secure the sealing component 410 to the shaft section 414, such as by insertion of the front pin 412 through an opening in the sealing component 410 prior to entry of the front pin 412 into the retention hole 416 of the shaft section 414.

In some instances, a space is formed between at least a portion of the sealing component 410 and at least a portion of the lumen section 406 when the sealing component 410 is secured to the lumen section 406. This can contribute to the flexibility of the sealing component 410 for creating the seal between the lumen section 406 and the lumen 502 (e.g., reducing the frictional force therebetween while still maintaining the seal). FIG. 19 illustrates a space 504 formed between the sealing component 410 and the front pin 412 when the front pin 412 is inserted through the sealing component 410.

One will appreciate, in view of the present disclosure, that other methods for securing the sealing component 410 to the lumen section 406 may be implemented in accordance with the present disclosure (e.g., adhesive, mechanical fit, threaded connection, etc.).

As noted above, a sealing component of a plunger may take on various forms. FIGS. 20 and 21 show an alternative form of a sealing component. FIG. 20 illustrates a plunger 602 where the sealing component is implemented as an O-ring 604, which may be coated (e.g., with an inert, lubricating material). The lumen section 606 of the plunger 602 includes a circumferential depression 608 configured to receive the O-ring 604. FIG. 21 shows the O-ring 604 interfacing with a lumen 702 to form a seal between the lumen section 606 and the lumen 702. In some instances, an O-ring design may require more force than a polymer sleeve design to facilitate a seal between the plunger and pipette lumen (e.g., in view of the lack of an interior space in the O-ring design).

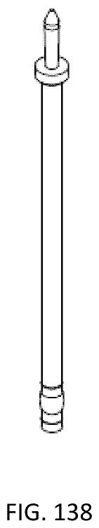

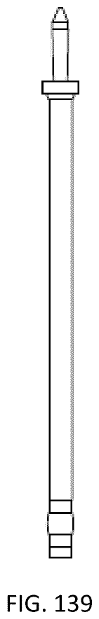

FIGS. 138-139 show an example of a pipette tip plunger having tapered engagement section for improved interaction with the gripping jaw of the pipette.

### Multichannel Pipette

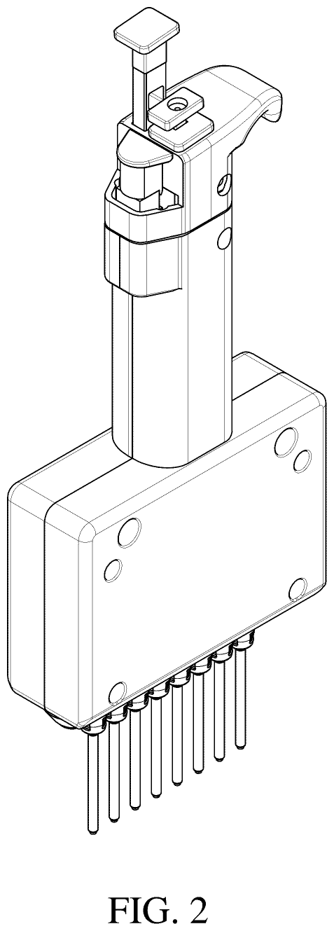

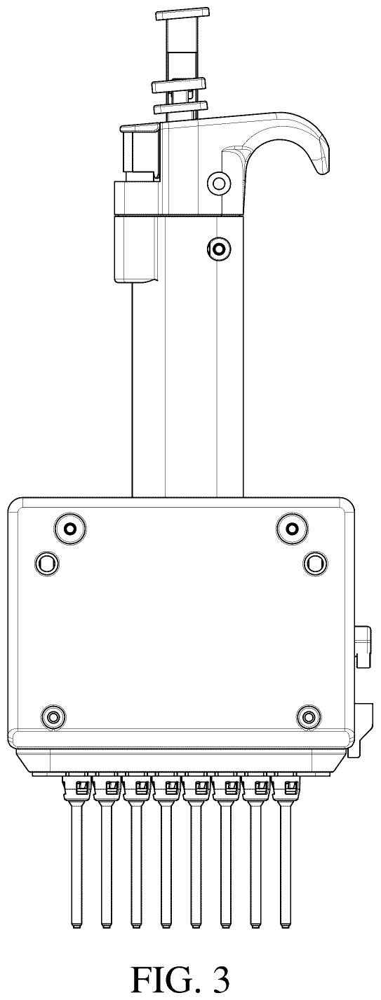

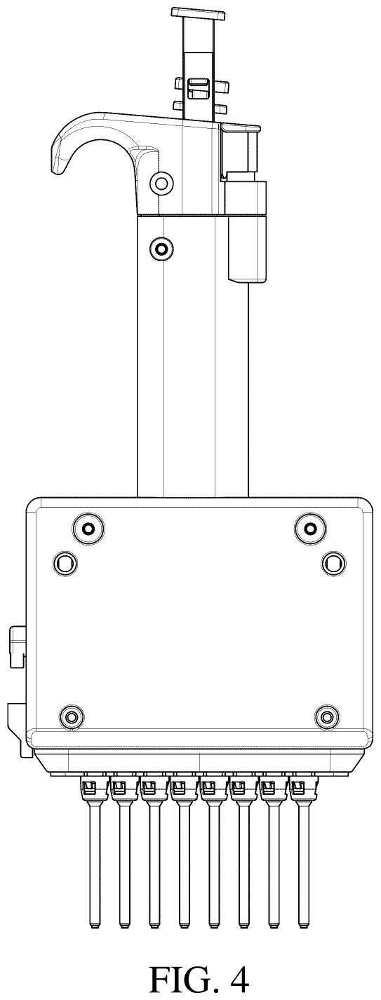

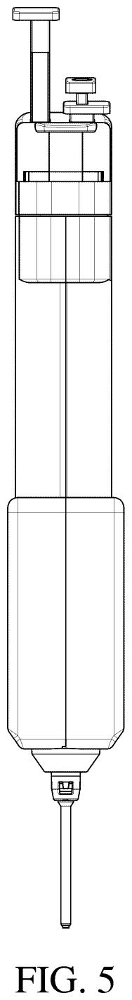

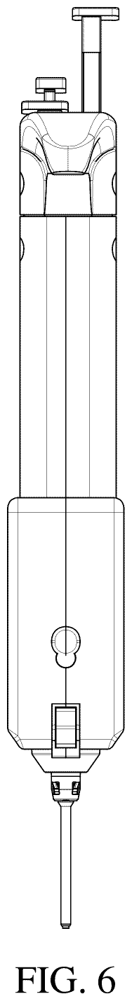

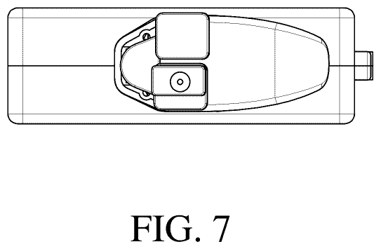

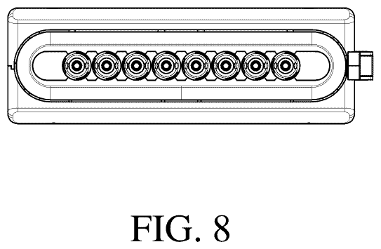

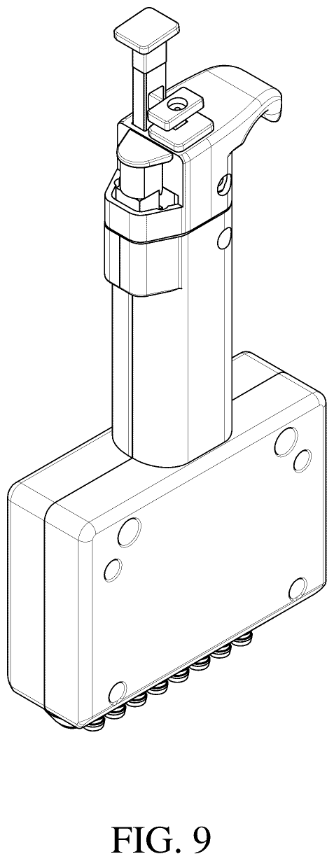

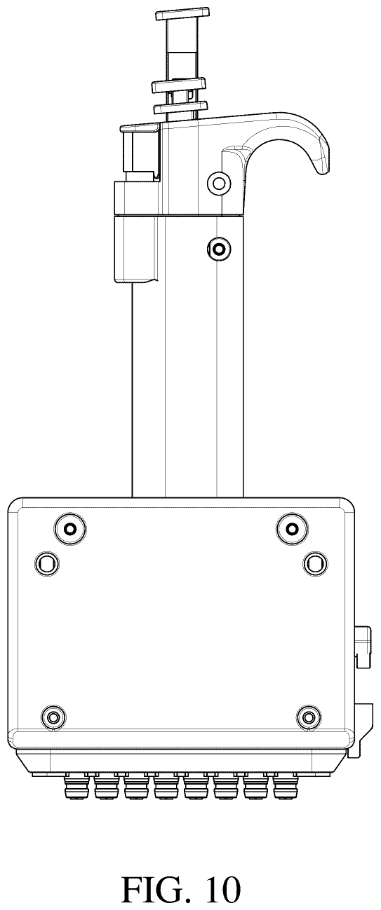

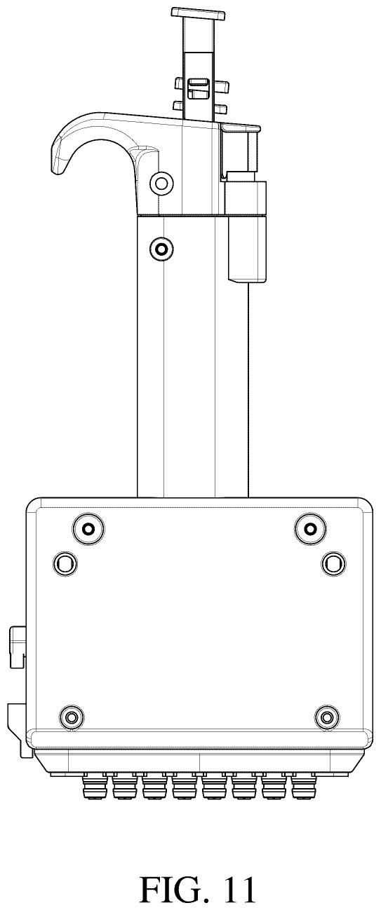

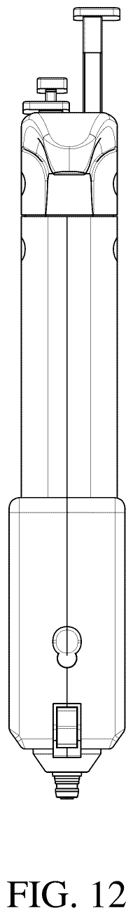

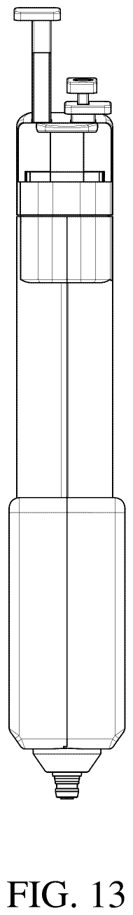

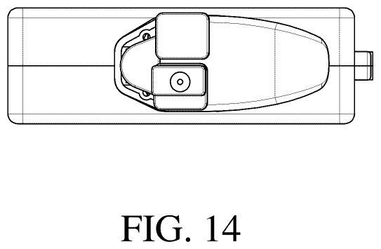

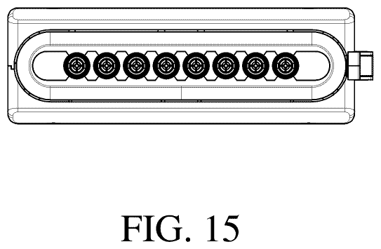

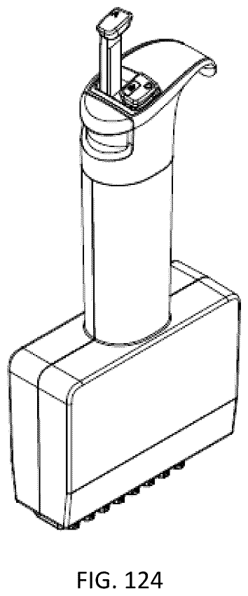

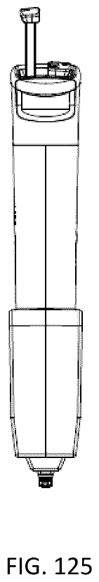

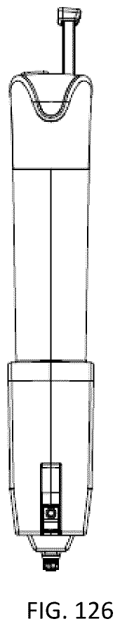

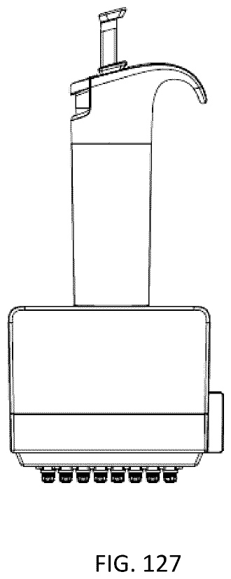

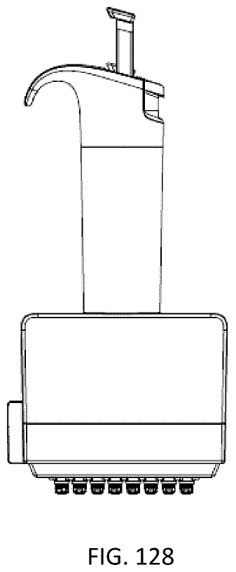

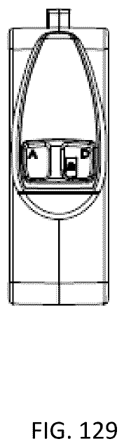

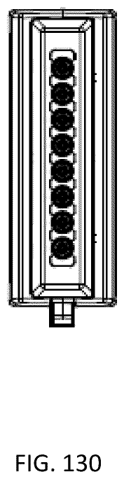

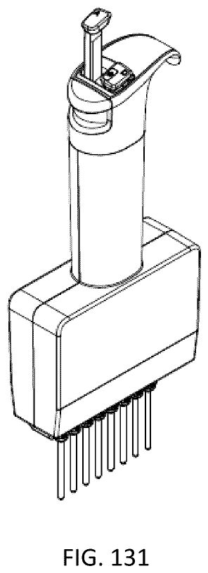

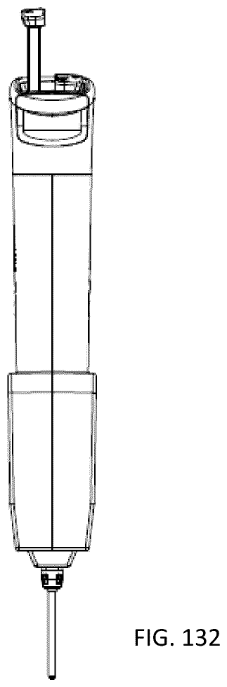

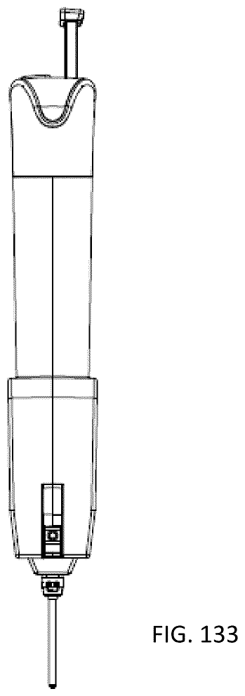

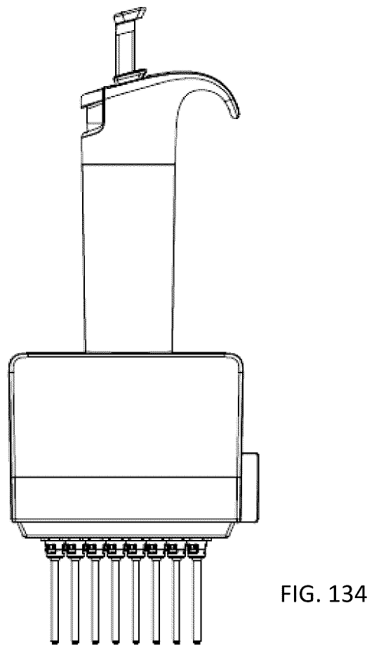

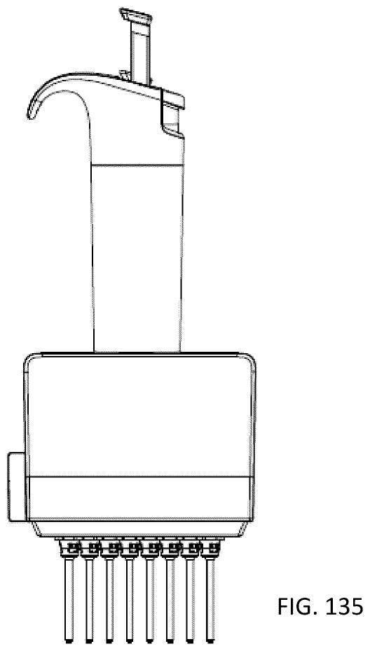

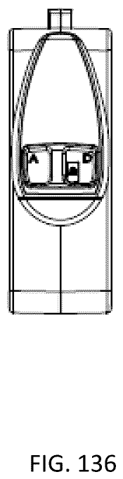

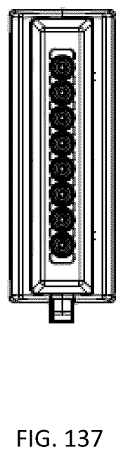

FIGS. 2-15 and 124-137 illustrate a multichannel pipette 130 utilized in the system 10 of the disclosure. It will be appreciated that while the present disclosure illustrates an embodiment of a pipette 130 configured to couple with up to 8 pipette tips to process samples, the pipette 130 of the disclosure may be configured to couple with 2, 3, 4, 5, 6, 7, 8, 9, 10 or more pipette tips 200 while maintaining the same functionality. In embodiments, the multichannel pipette 130 includes: a proximal section 135 having a handle 140, a distal section 145 configured to reversibly engage a plurality of pipette tips 200, a first actuator 150 disposed in the proximal section 135, and a second actuator 155 disposed in the proximal section 135 operable to control dispensing function of the multichannel pipette 130. In various embodiments, the first actuator 150 is operable to control aspiration and the second actuator 155 is operable to control dispensing. In embodiments, the pipette further includes a third actuator 160 disposed in the proximal section 135 configured to cause a pipette tip 200 attached to the distal section of the multichannel pipette 130 to disengage when the third actuator 160 is actuated, as well as a lock button 165 disposed in the proximal section 135. It will be appreciated that the actuators (150, 155 and 160) and the lock button 165 are oriented such that operation of the respective functions of each are controllable by the thumb of a user grasping the handle 140 and operating the pipette 130. The pipette 130 also includes a pipette electrode 132 which contacts an electrode disposed on the docking station when the pipette is docked in the pipette docking assembly (see FIG. 3).

In various embodiments, during operation of the pipette 130, the first actuator 150 controls aspiration of fluid into pipette tip(s) 200 attached to the distal section 145 of the pipette 130. The second actuator 155 controls dispensing of fluid from attached pipette tip(s) 200. The lock button 165 functions to activate a locking mechanism disposed in the distal section 145 of the pipette to grip and lock the plunger lumenally disposed within each pipette tip 200 during an electroporation protocol including aspiration, electroporation, and dispensing of sample. The third actuator 160 functions to disengage the lock mechanism and simultaneously dislodge pipette tip(s) 200 attached via clip-on connection(s) from the pipette 130 as discussed further herein.

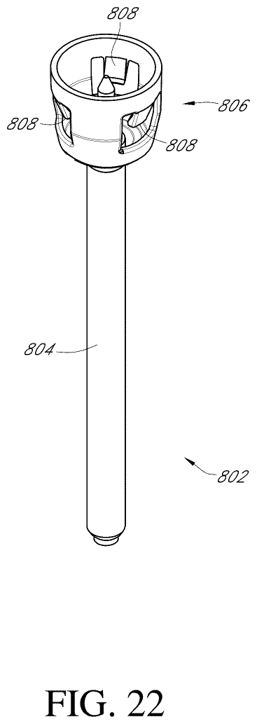

As noted above, one or more pipette tips may be selectively attached to the pipette 130 via a clip-on connection. FIG. 22 illustrates a pipette tip 802, which corresponds to a pipette tip shown in FIGS. 16 and 17 and including features that enable a clip-on type connection with the pipette. As illustrated in FIG. 22, the clip-on connection includes an attachment interface 806 adjacent to the lumen 804 of the pipette tip 802. The attachment interface 806 includes tabs 808, which are configured to engage with corresponding attachment features of the pipette 130 (any number of tabs may be utilized). The tabs may be angled toward the lumen of the pipette tip 802. The corresponding attachment features of the pipette 130 may be arranged in the distal section 145 of the pipette 130 about individual gripper mechanisms which are discussed in detail further herein.

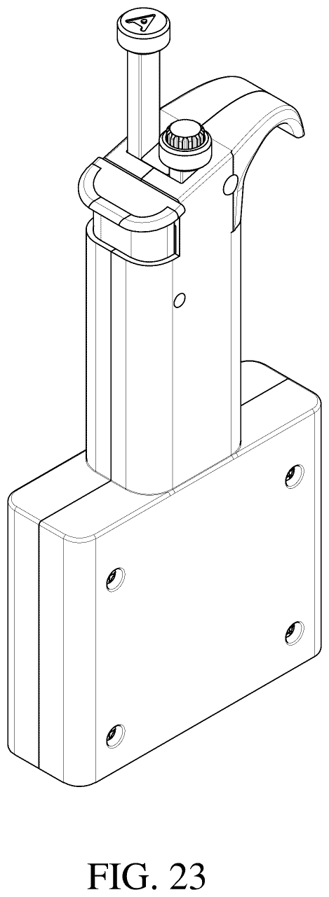

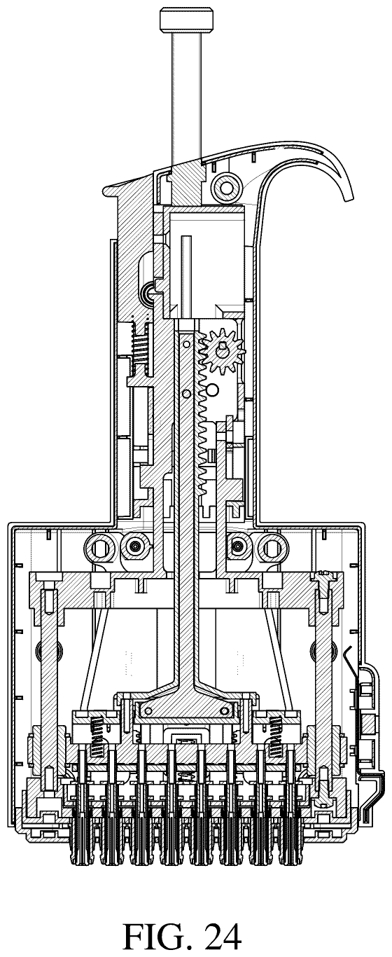

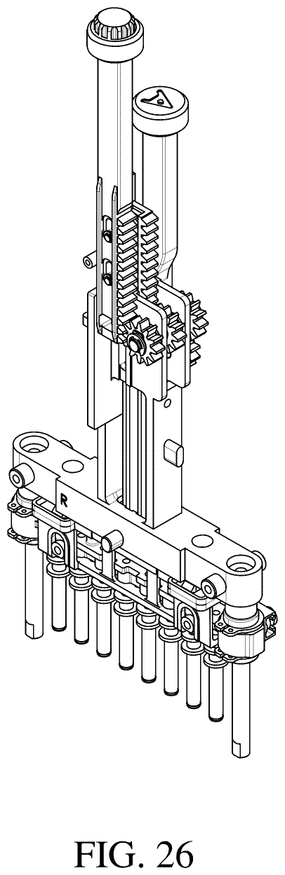

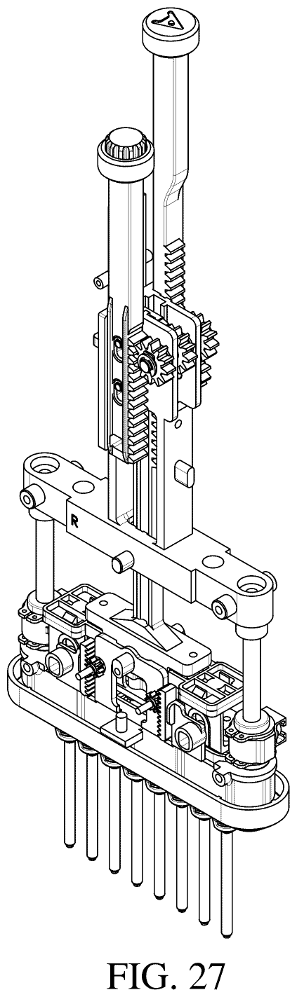

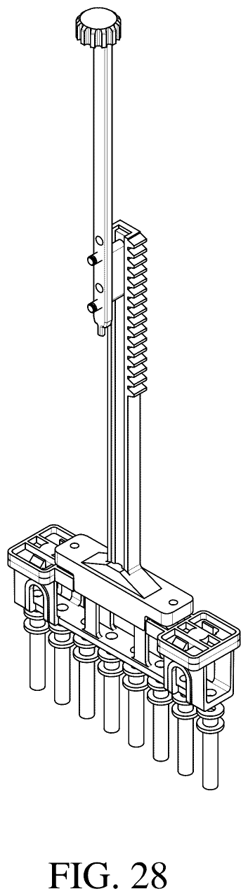

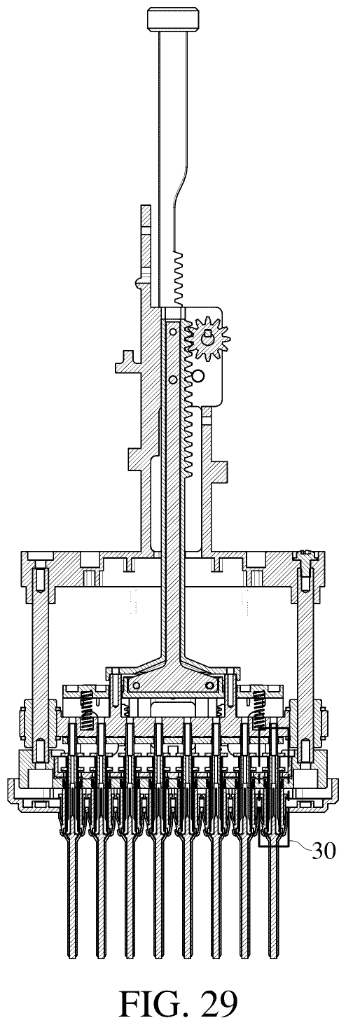

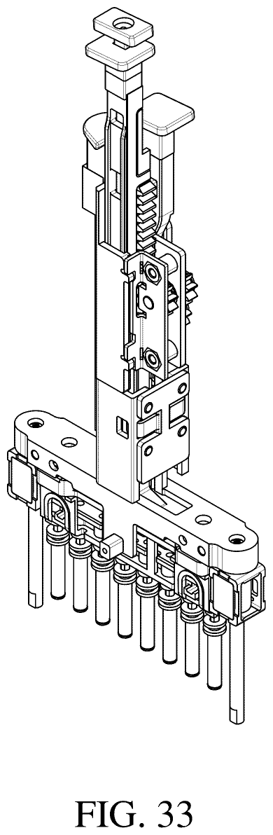

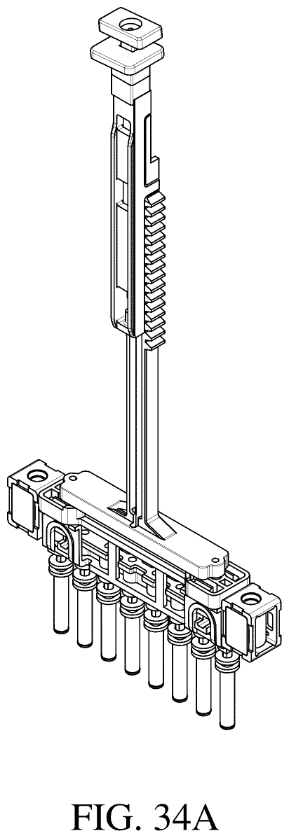

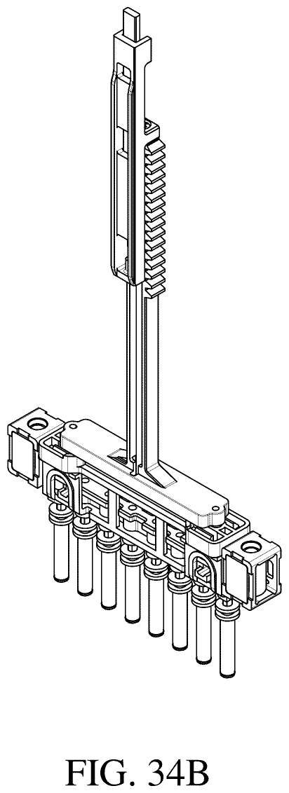

Operation of the pipette 130 to perform aspiration, dispensing and locking of pipette tips is shown with reference to FIGS. 23-36. In embodiments, the first actuator 150 is configured to transition from a first undepressed position to a second depressed position to aspirate fluid into an attached pipette tip (see FIGS. 26 and 33). Additionally, the second actuator 155 is configured to transition from a first depressed position to a second undepressed position when the first actuator 150 is transitioned from the first undepressed position to the second depressed position such that fluid contained in the pipette tip can be dispensed after being electroporated by transitioning the second actuator 155 from the second undepressed position to the first depressed position. Transitioning the first actuator 150 from the first undepressed position to the second depressed position causes an aspiration function (e.g., aspiration of fluid into a pipette tip attached at the distal section of the pipette) and transitioning of the second actuator 155 from the second undepressed position to the first depressed position causes a dispensing function (e.g., dispensing of fluid from a pipette tip attached at the distal section of the pipette).

In various embodiments, the pipette includes a lock button 165 disposed in the proximal section 135 of the pipette as shown in, for example, FIGS. 2, 9, 23 and 31. The lock button 165 is operably connected to a lock mechanism configured to control locking of the plurality of gripper mechanisms as discussed in detail further herein. Depressing the lock button causes the lock mechanism to transition from a first unlocked configuration to a second locked configuration. When the lock mechanism is in the first configuration the plurality of gripper mechanisms are open and configured to receive the engagement section of the plunger and when the lock mechanism is in the second configuration the plurality of gripper mechanisms are closed and configured to be in grasping engagement with the engagement section of the plunger of the pipette tip. In some embodiments, the lock mechanism is transitioned from the second locked configuration to the first unlocked configuration when the third actuator 160 is actuated (e.g., depressed).

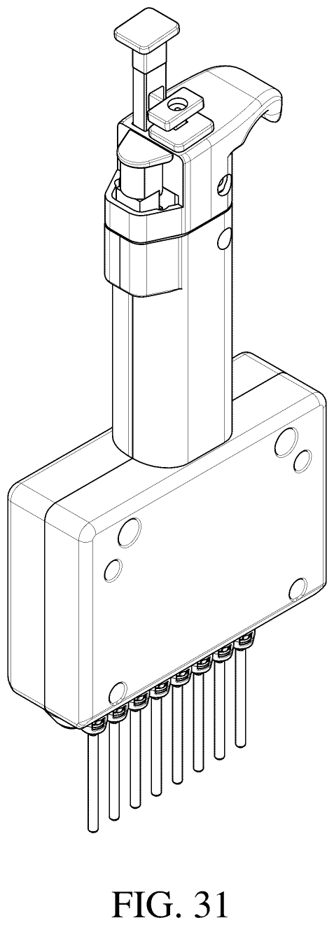

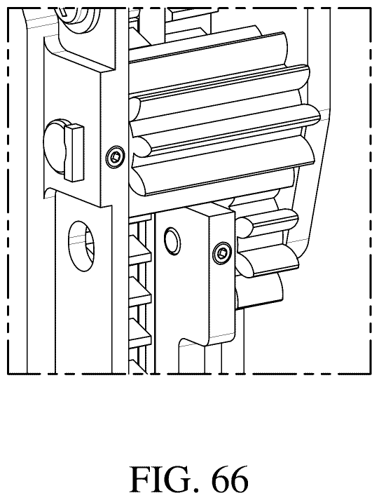

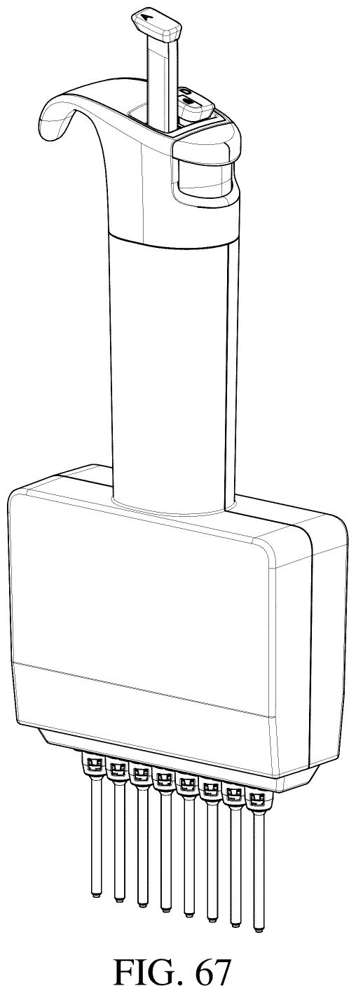

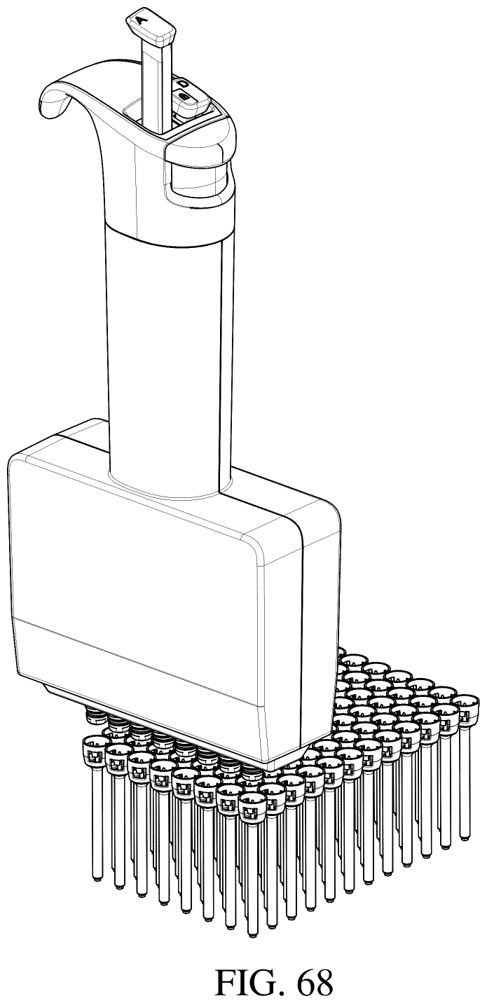

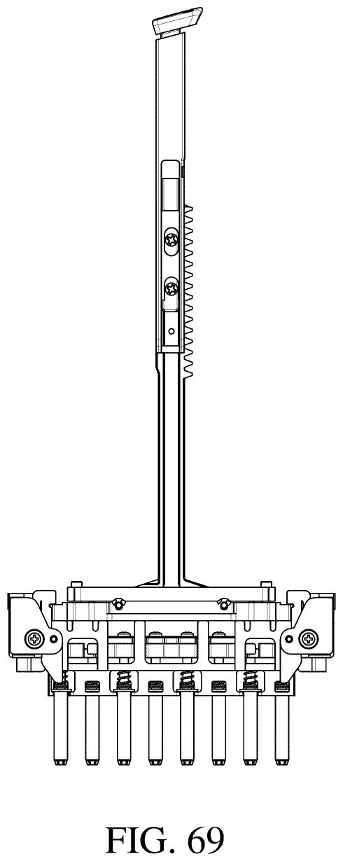

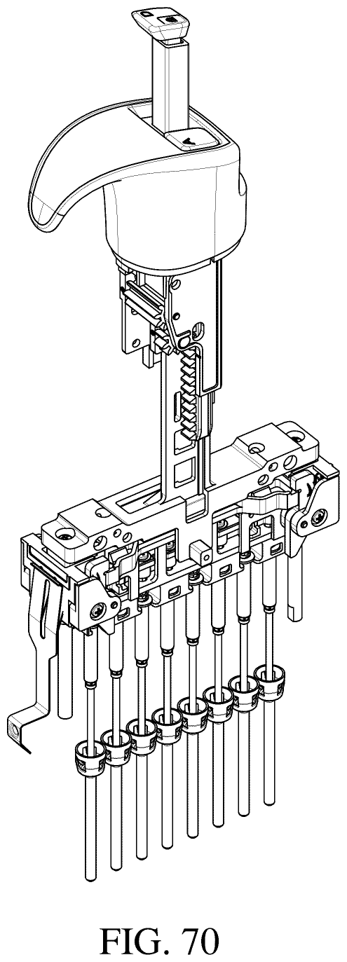

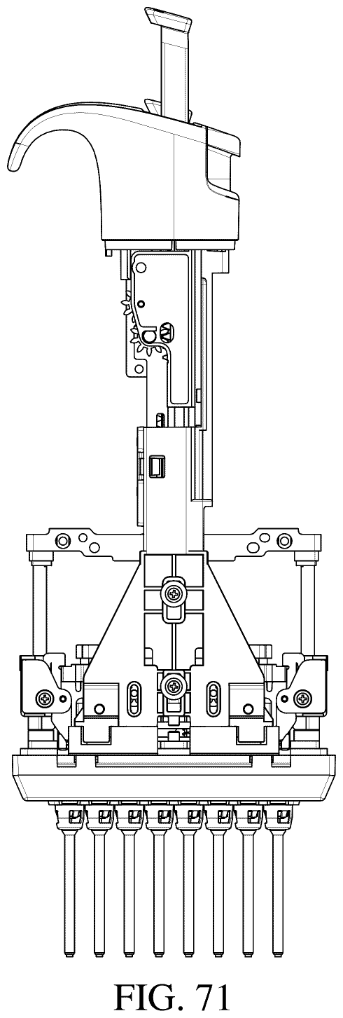

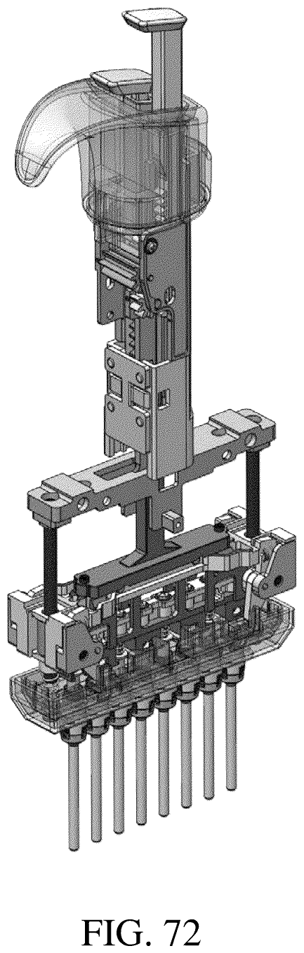

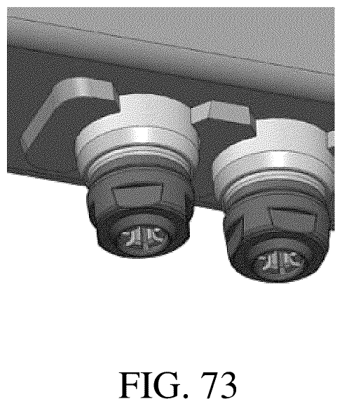

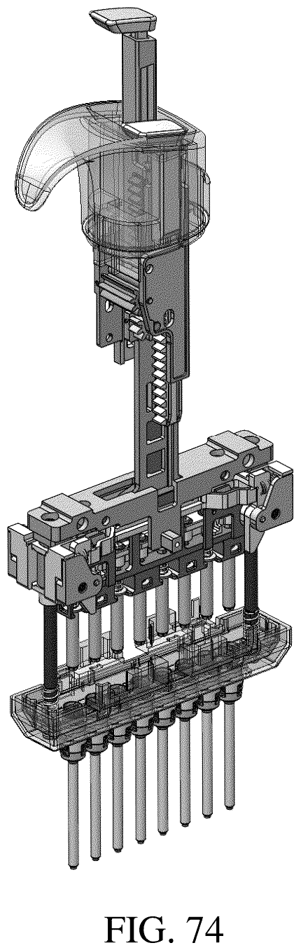

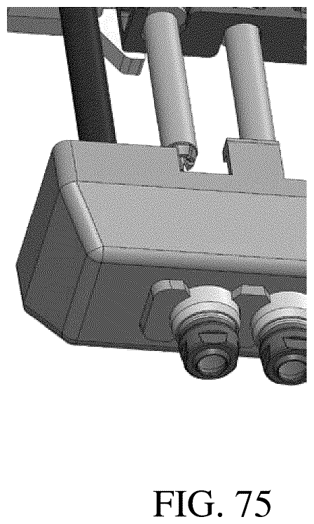

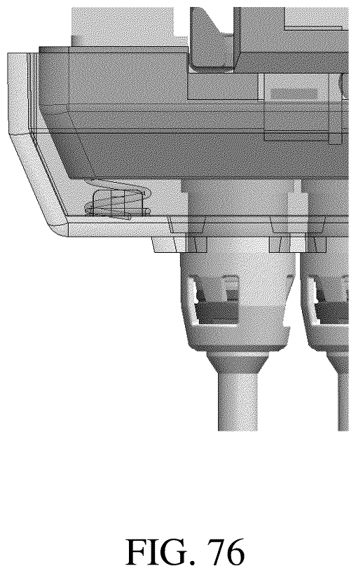

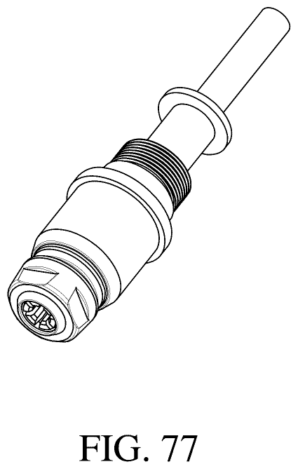

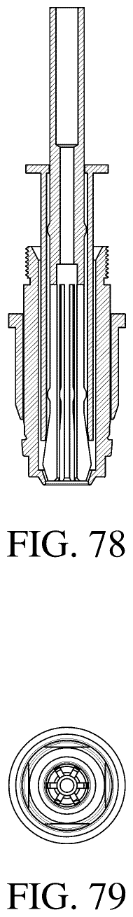

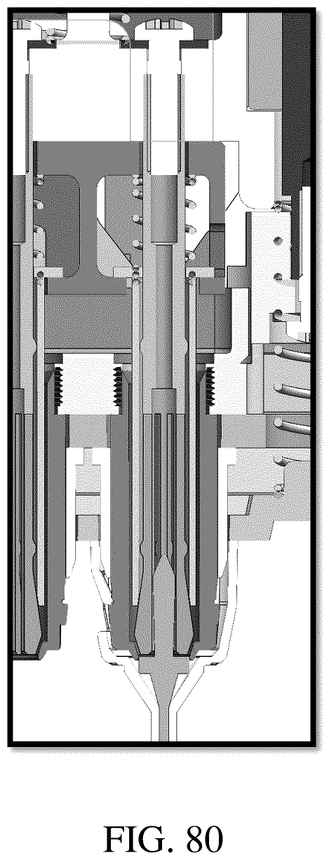

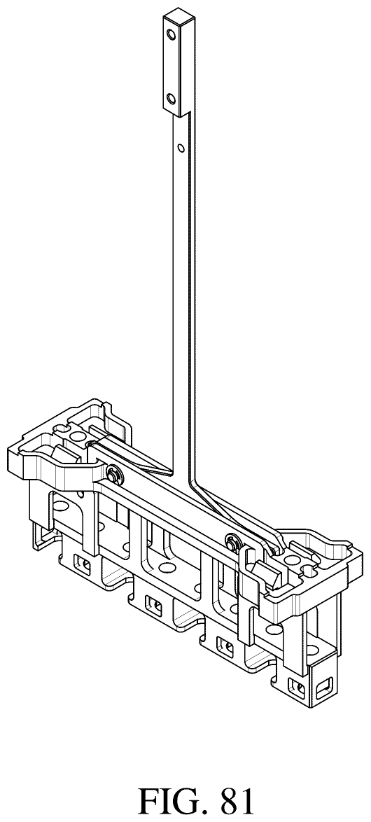

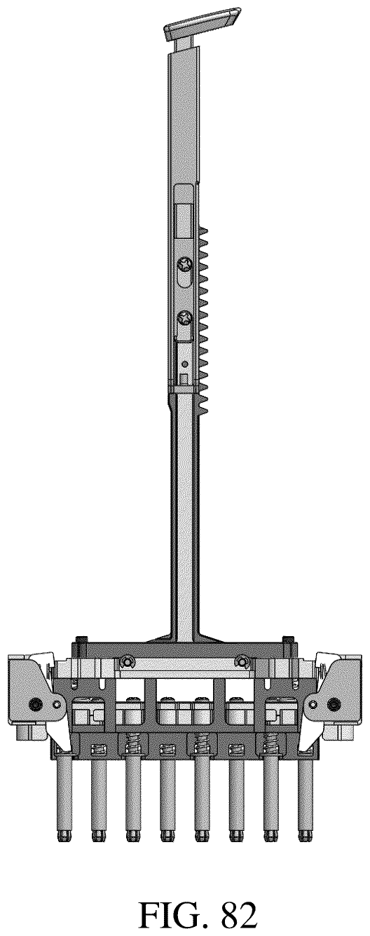

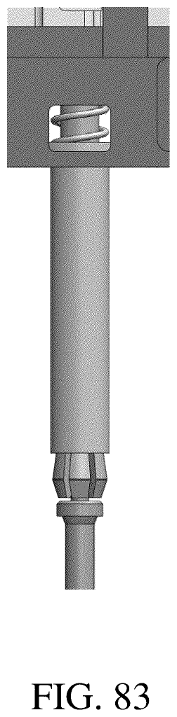

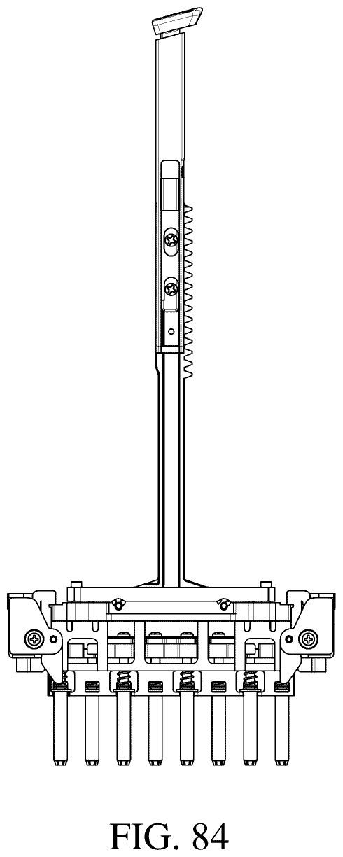

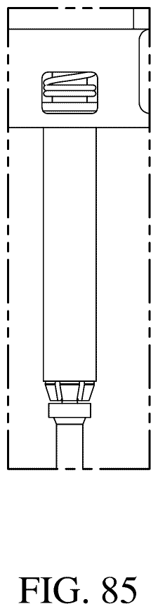

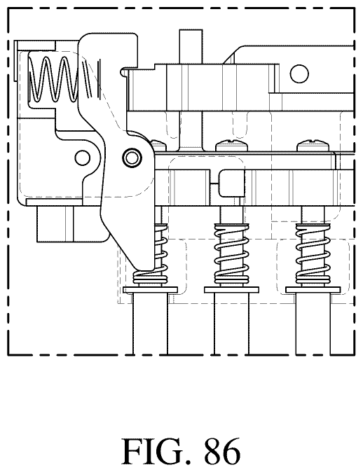

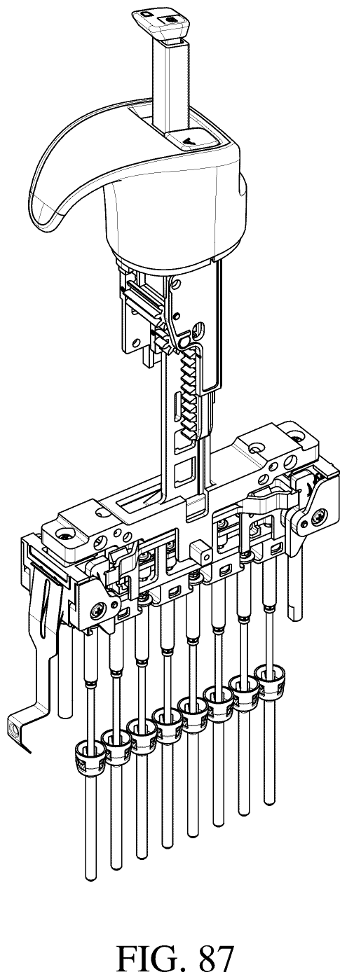

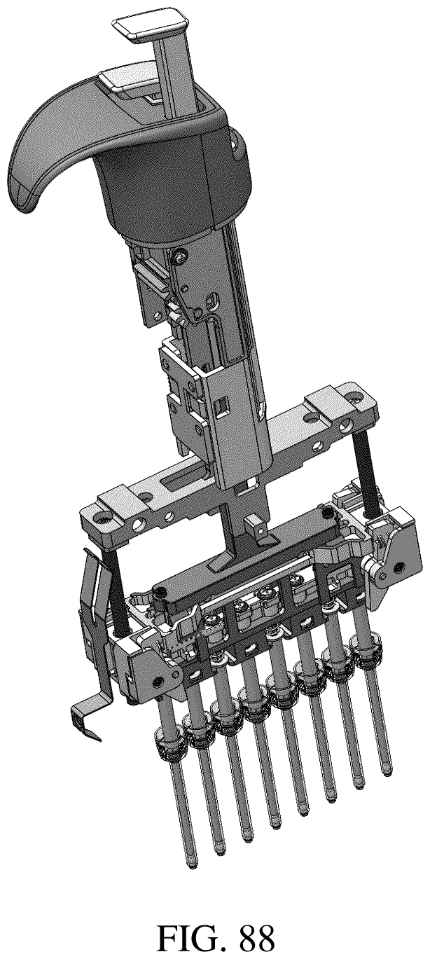

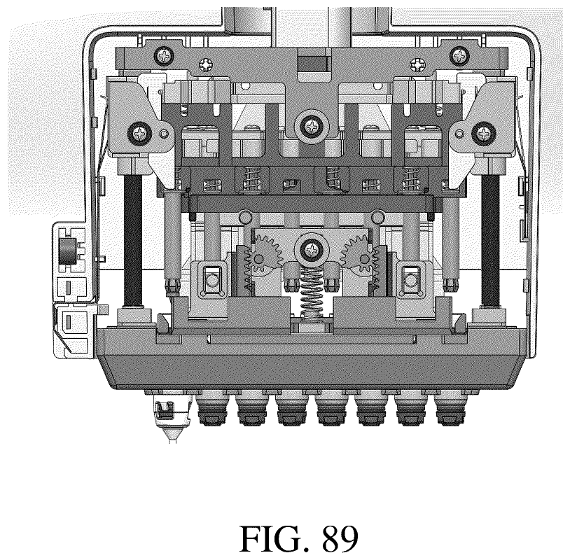

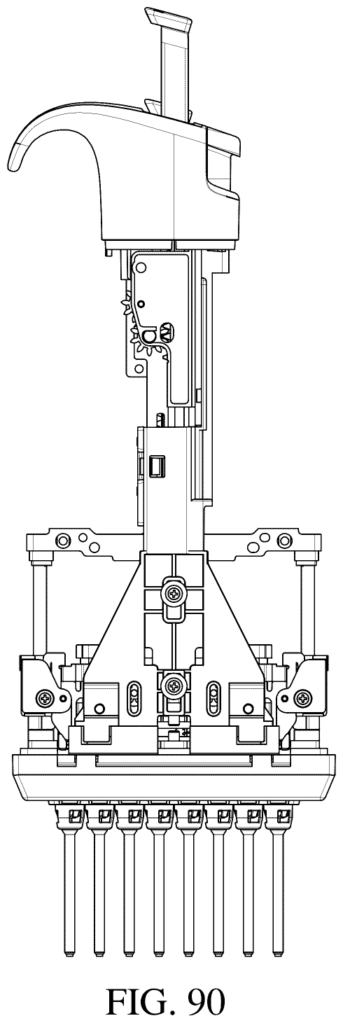

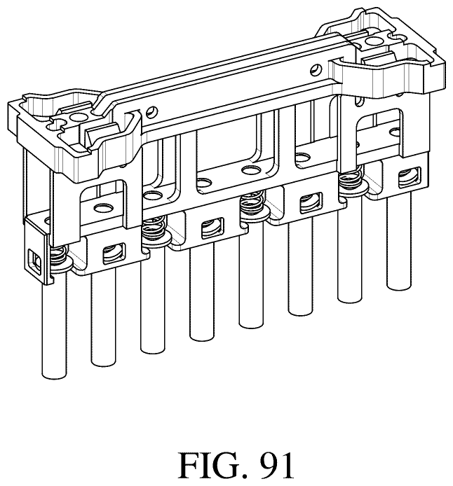

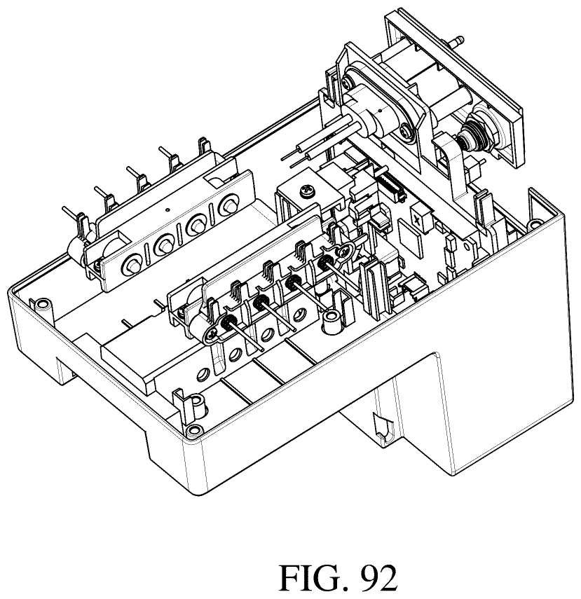

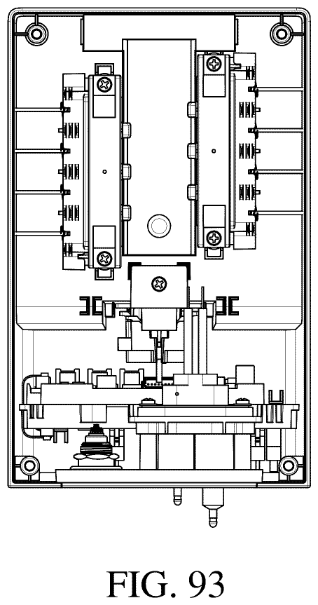

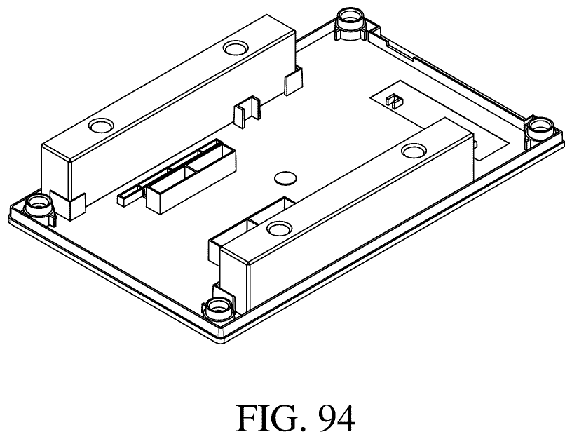

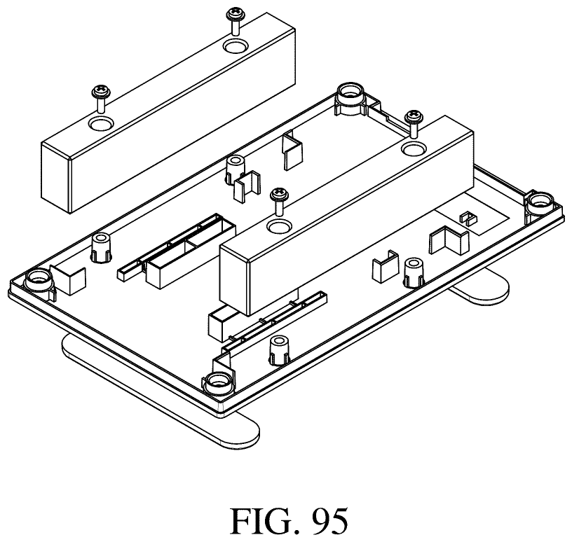

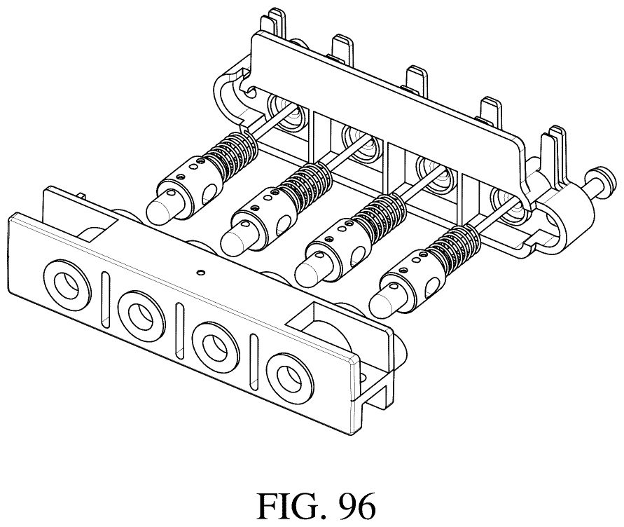

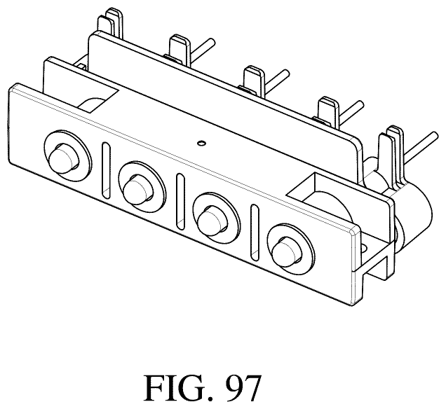

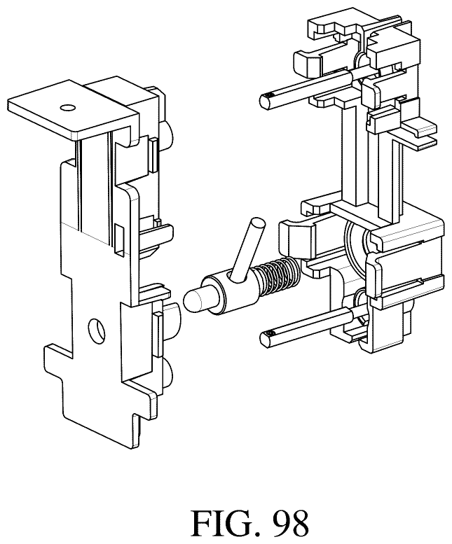

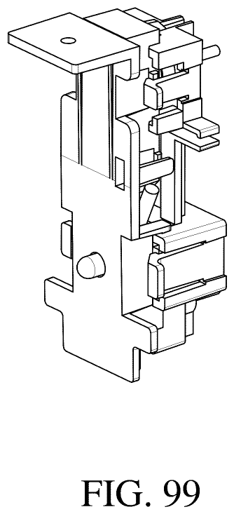

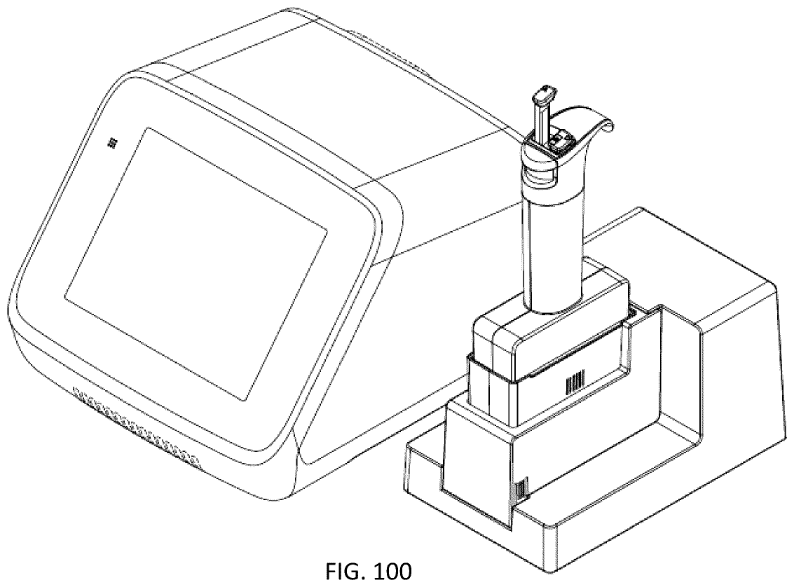

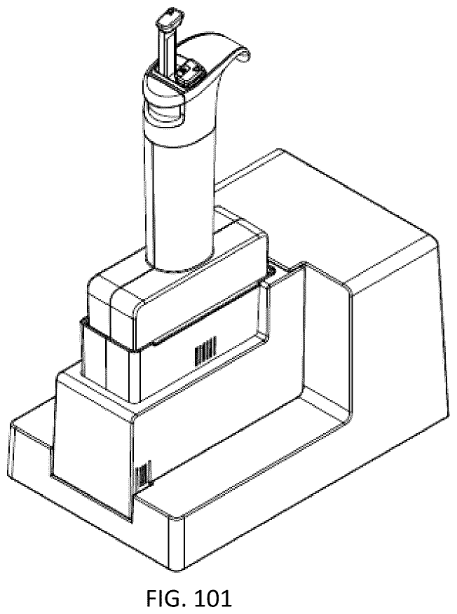

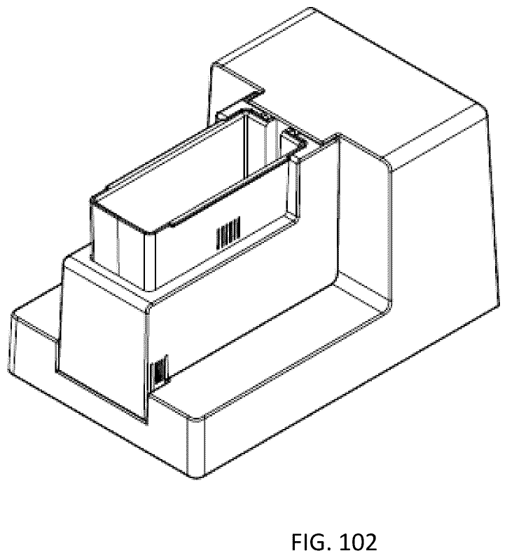

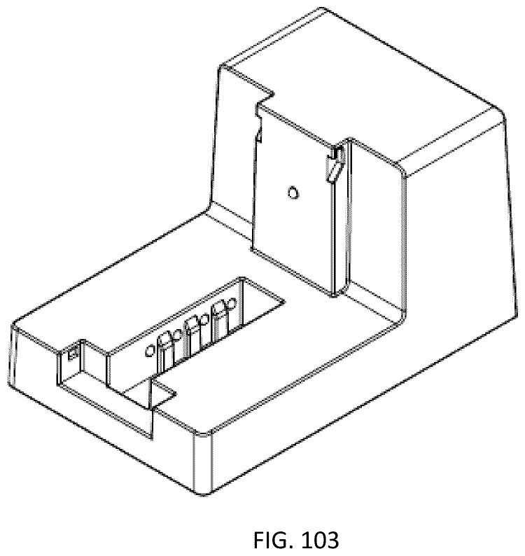

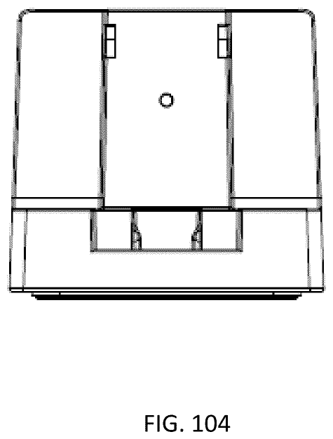

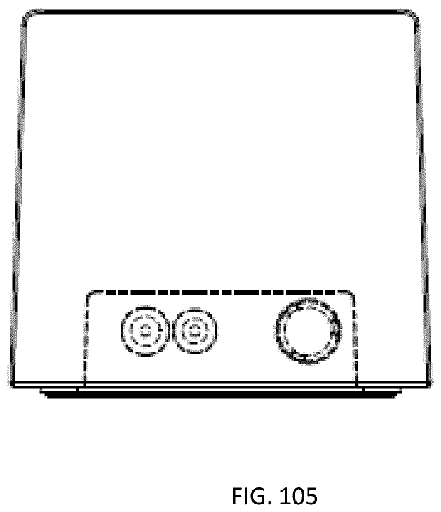

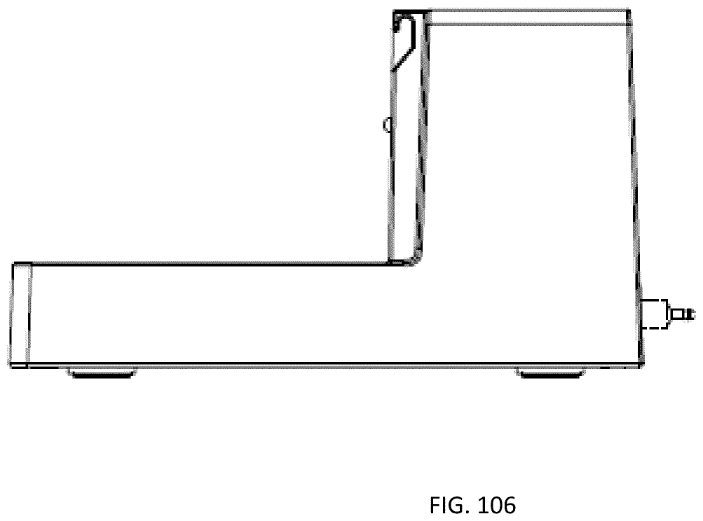

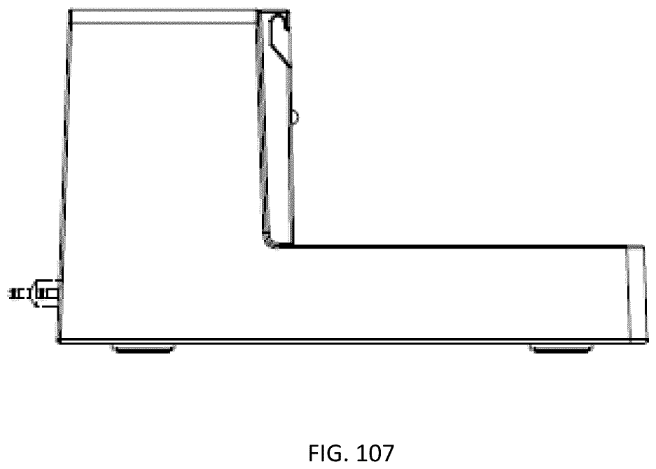

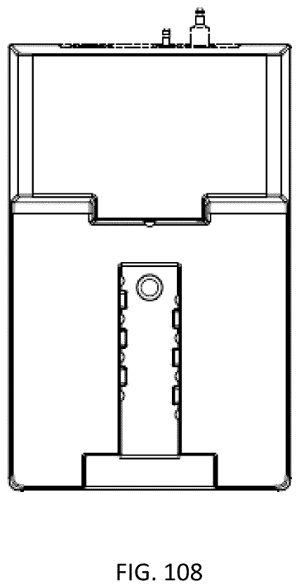

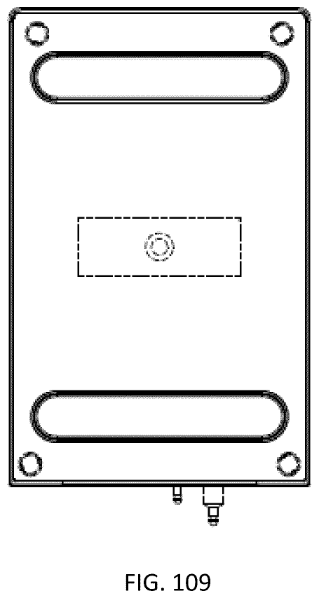

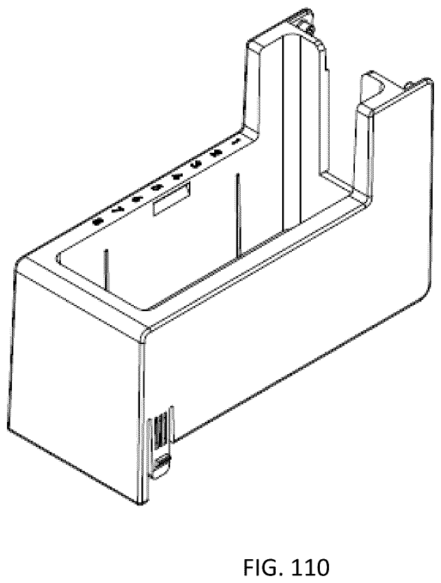

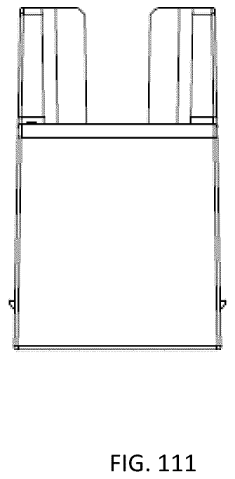

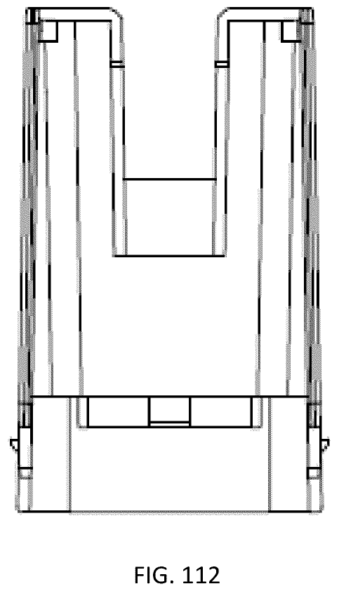

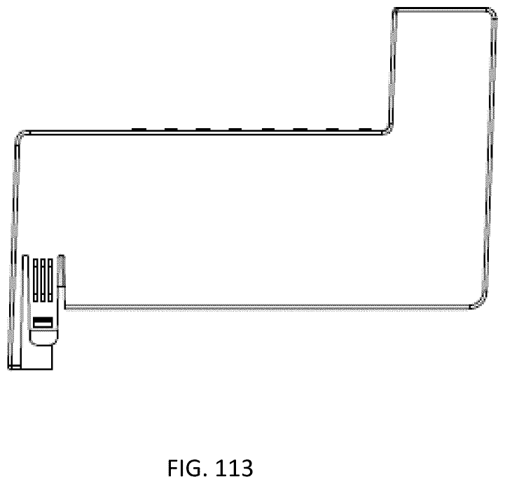

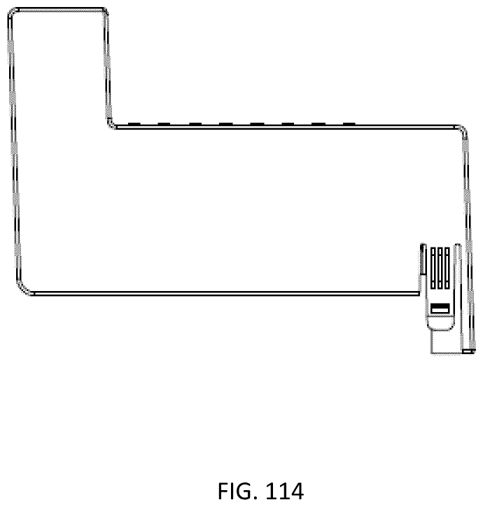

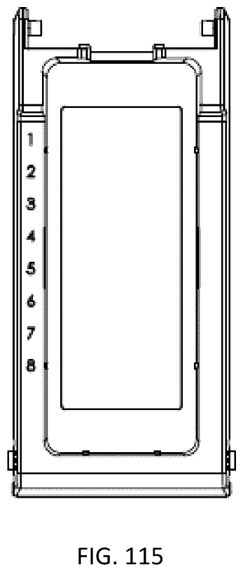

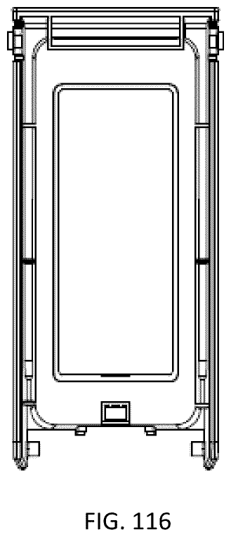

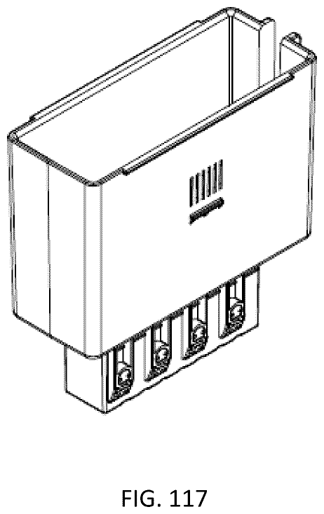

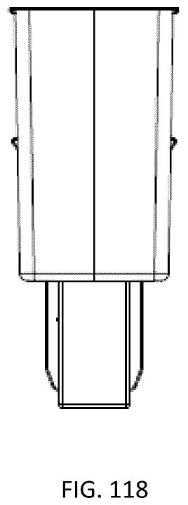

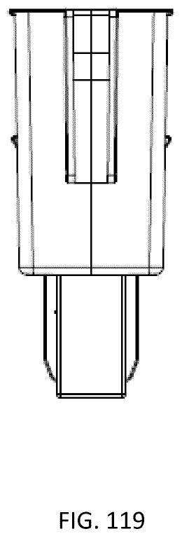

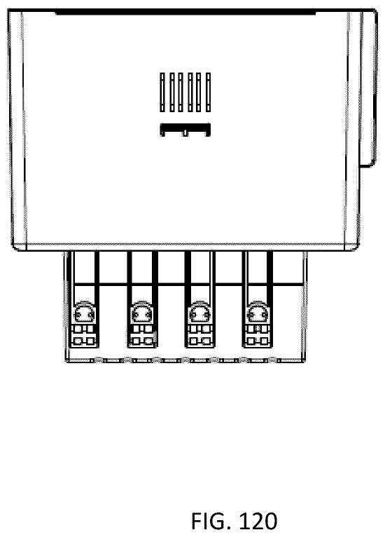

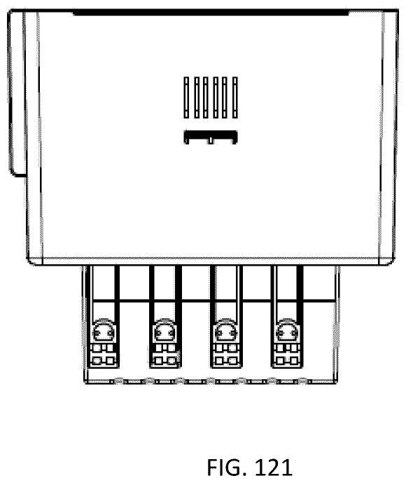

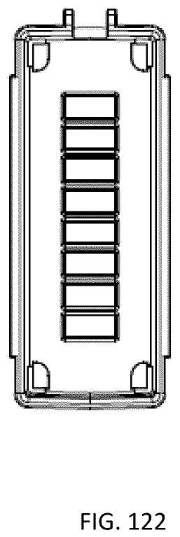

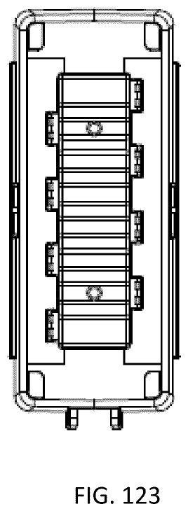

FIGS. 23-30 show an embodiment of the pipette 130 which includes a gear mechanism 170 having a particular configuration of operably connected gears. FIGS. 31-36 show another embodiment of the pipette 130 which includes a gear mechanism 170 having a different configuration of gears in comparison to the gear mechanism 170 shown for the pipette 130 of FIGS. 23-30. However, both gear mechanism configurations provide similar aspiration and dispensing functionality and are achieved by the same manual operation of the first actuator 150, the second actuator 155, the third actuator 160 and the lock button. Additionally, FIG. 66 shows another embodiment of the gear mechanism 170 shown for the pipette 130 of FIGS. 31-36 in which gears are fixed to a single shaft to allow frictionless gear rotation.

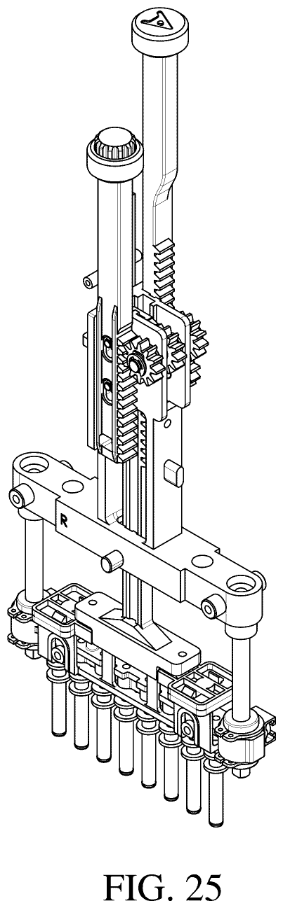

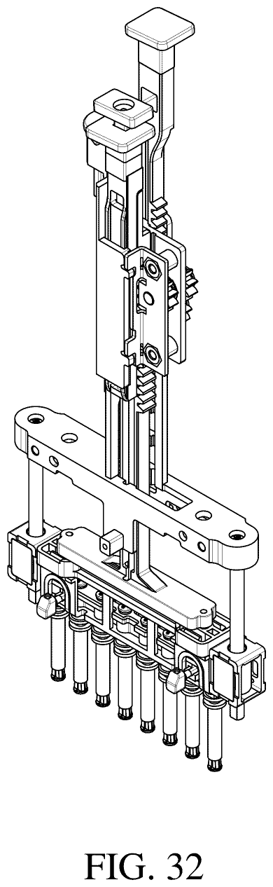

FIGS. 25 and 32 illustrate the movement of individual gears in the transitioning of the second actuator 155 to the first depressed position to dispense fluid. FIGS. 26 and 33 illustrate the movement of individual gears in the transitioning of the first actuator 150 to the second depressed position to aspirate fluid.

Additional components of the pipette 130 are shown throughout FIGS. 23-36. In various embodiments, the pipette 130 includes shafts 175, a linkage rack 174, a push plate 176, a linkage bar 178, bushings 180, and a plurality of gripper mechanisms 182, each gripper mechanism having a gripper jaw 184 movably disposed within a gripping sleeve 186. The linkage bar 178 is rigidly connected to individual gripper jaws 184 and the push plate 176 which operates to contact a proximal end of each gripping sleeve 186. The linkage rack 174 is operably connected to the push plate 176 and translates movement of the first and second actuators (150, 155), as well as depressing of the lock button 165, to the linkage bar 178 during performance of aspiration, dispending and locking functions. The bushings 180 are operably connected to the linkage bar 178 and engage the shafts 172 such that there is minimal resistance during linear movement of the linkage bar 178 along the length of the shafts during operation. This allows for highly accurate and supple movement to be achieved.

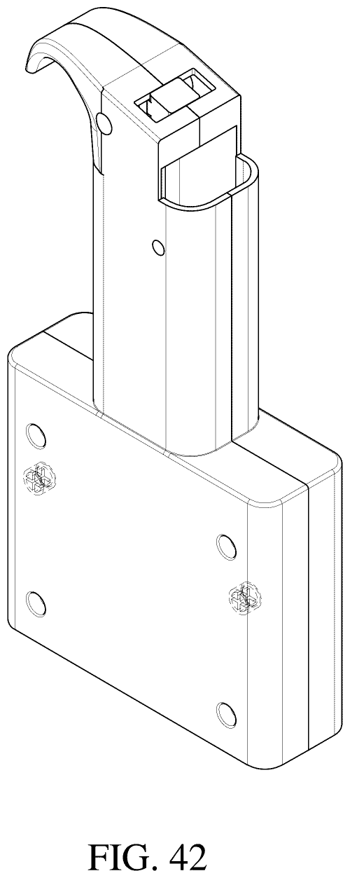

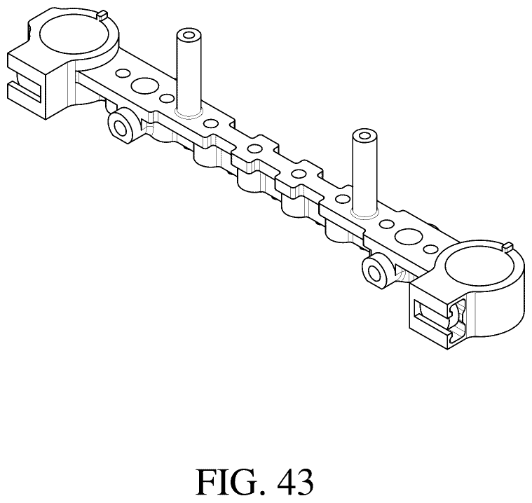

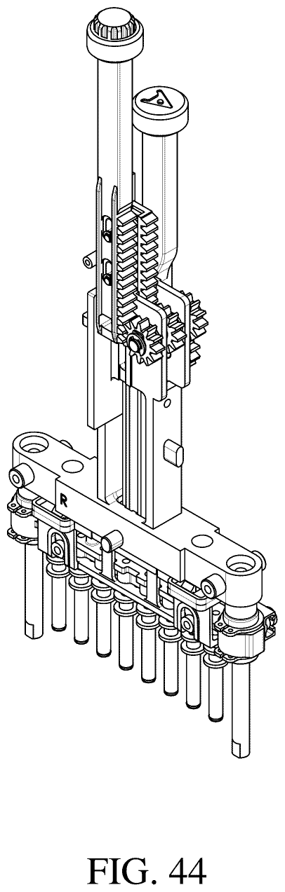

With reference to FIGS. 42-44, in embodiments, the pipette 130 also includes magnets that are configured to interact with each other to enable certain functionality. In various embodiments, the pipette 130 includes an outer housing 900 that includes discrete magnets 910 that interact with discrete magnets 910 disposed on internal components of the pipette, the magnetic interaction of which, achieves a particular function. FIGS. 42-44 illustrate portions of the pipette depicted in FIGS. 2-15 and 31-36. In such embodiments, the outer housing 900 includes magnets 910 that are disposed into or on the outer housing such that they interact with magnets disposed into or on the linkage bar 178 of the pipette. FIG. 42 shows the location of the magnets disposed on the outer housing of the pipette which magnetically interact with magnets disposed on the linkage bar 178 as shown in FIG. 43. As shown in FIG. 44, after performing an aspiration function of the pipette (e.g., moving the first actuator from the first undepressed position to the second depressed position), the linkage bar 178 is magnetically held in a position such that the push plate 176, bushings 180 and components of the gripper mechanisms (e.g., gripper jaw 184 and gripping sleeve 186) are oriented toward the proximal section of the pipette along the length of the shafts. In this configuration, a majority portion of the lengths of individual plungers of attached pipette tips held within gripper jaws 184 are also withdrawn from the lumens of the pipette tips which contain aspirated fluid. The pipette 130 is held in this configuration while the pipette is inserted into the pipette docking assembly 110 and the electroporation procedure is performed.

With reference to FIGS. 25 and 32, the pipette described herein includes a plurality of gripper mechanisms 182 disposed in the distal section 145 of the pipette. In embodiments, each gripper mechanism 182 is configured to engage components of a pipette tip 200 suitable for performing electroporation. The pipette tip is first attached to the distal section of the pipette via a clip-on connection formed by engagement of tabs of the attachment interface of the pipette tip with attachment features surrounding portions of the gripper mechanism. Next, a plunger of the pipette tip is grasped and locked by the gripper mechanism which allows linear movement of the plunger within the lumen of the pipette tip to be controlled via manipulation of the first and second actuators and lock button of the pipette to perform pipetting functions.

As discussed herein, pipette tips for use with the pipette of the disclosure each include a clip-on connection along with a plunger disposed within a lumen of the pipette tip which is composed of electrically conductive material to facilitate flow of electrical current through a sample contained in the lumen of the pipette tip during use to perform a cell transfection protocol.

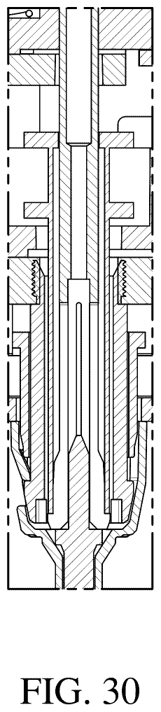

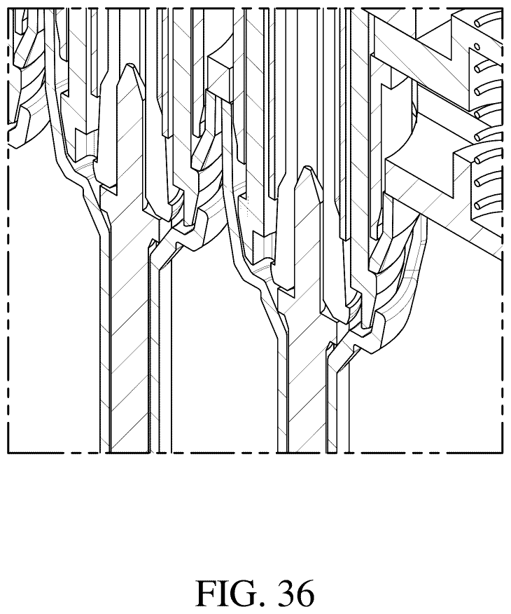

As shown in FIGS. 30 and 36, individual gripper mechanisms are configured to reversibly grip the plunger 402 of a pipette tip 200 via the engagement section of the plunger once the pipette tip is attached to a gripper mechanism via a clip-on connection. When the engagement section of the plunger is gripped by the gripper mechanism and the lock mechanism is actuated (e.g., in the second locked configuration via depressing the lock button), movement of the plunger 402 within the lumen of the pipette tip is controlled by the first and second actuators to perform pipetting functions, e.g., aspiration and dispensing of fluid into and out of the lumen of the pipette tip.

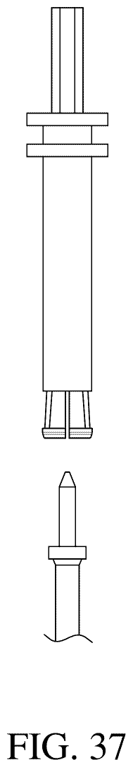

FIGS. 37-41 illustrate components of a gripper mechanism in some embodiments. Each gripper mechanism 182 includes a gripper jaw 184 which has a jaw opening 185 for receiving and retaining the engagement section 404 of the plunger, and a gripping sleeve 186 positioned around the gripper jaw. The gripping sleeve 186 is configured to exert an inward force on the gripper jaw 184 to cause the gripper jaw to exert an inward force on the engagement section of the plunger to retain the engagement section of the plunger within the gripper jaw when the lock button is depressed.

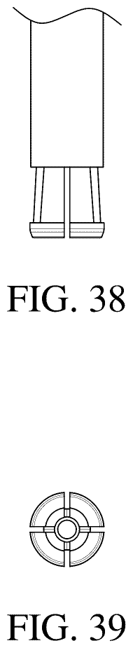

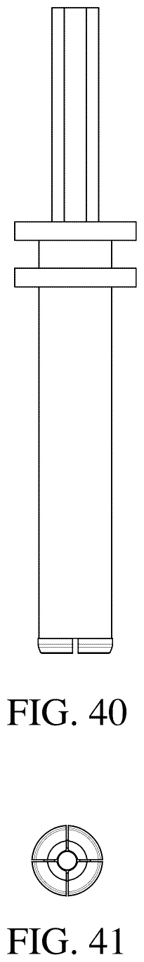

FIGS. 38 and 39 show the gripper mechanism 182 in an open configuration suitable to receive the engagement section 404 of the plunger. FIGS. 40 and 41 show the gripper mechanism 182 in a closed configuration suitable to grasp the engagement section 404 of the plunger. Notably, distally oriented movement of the gripping sleeve 186 relative to the gripper jaw 184 translates the gripper mechanism 182 from the open configuration to the closed configuration. This movement is achieved by depressing the lock button.

FIG. 36 is a cross sectional view of a gripper mechanism grasping the engagement section of a plunger of a pipette tip attached to the tip interface via the attachment interface of the pipette tip. Surrounding the gripping sleeve 186 is a tip interface 188 which is configured to engage the attachment interface 806 (see FIG. 22) of a pipette tip and includes a retention platform 955 which engages tabs 808 (see FIG. 22) of the attachment interface to facilitate a clip-on connection. In embodiments, the retention platform 955 is disposed distally on the tip interface and circumnavigates the outer surface of the tip interface 188 as discussed further herein.

As shown in FIG. 36, the tip interface 188 includes an attachment feature, e.g., the retention platform 955, for engaging the tabs 808 of the attachment interface 806. FIG. 36 shows the retention platform 955 disposed on the outer surface of the tip interface 188 in the distal section of the pipette. The retention platform 955 resides atop a slanted surface 960 of the tip interface and is defined by a flat annular surface that traverses around the circumference of the tip interface 188, the surface extending radially from the longitudinal axis of the tip interface at a 90 degree angle such that the annular surface of the retention platform is perpendicular to the longitudinal axis of the tip interface and the longitudinal axis of the pipette. During attachment of the pipette tip, when the distal section 145 of the pipette 130 is pressed into the attachment interface 806 of the pipette tip, the tabs 808 of the attachment interface may advance and expand over the slanted surface 960 until reaching the retention platform 955, at which time the tabs 808 may retract inward toward the longitudinal axis of the tip interface and into engagement with the retention platform 955.

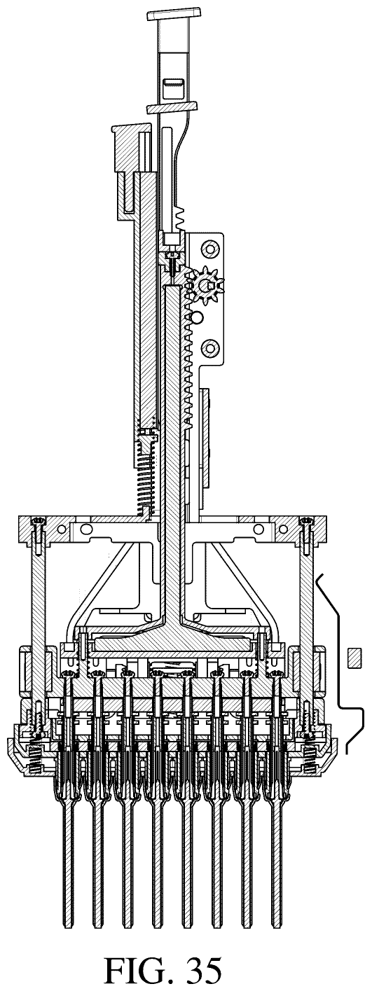

In some instances, after the tabs 808 reach the retention platform 955 and are retracted toward the central axis of the tip interface 188, a biasing member of the pipette 130 may operate to bias the tabs 808 into engagement with the retention platform 955. FIG. 35 shows pipette tips 200 attached to tip interfaces and a biasing member 962 of the distal section 145 of the pipette 130, which includes a biasing platform 964. Spring(s) 966 are operably connected to the biasing member 962 and function to perpendicularly align pipette tips with the biasing platform 964.

FIG. 36 shows the attachment interface 806 of the pipette tip engaged with the retention platform 955 of the gripping sleeve 186. During attachment of a pipette tip, as the attachment interface 806 advances over the tip interface 188, the attachment interface 806 presses and moves the biasing platform 964 to allow the tabs 808 to reach the retention platform 955. The spring 966 responsively becomes compressed such that after pressing of the attachment interface 806 into the distal section 145 of the pipette 130 ceases (after the tabs 808 have reached the retention platform), the spring forces the biasing platform 964 against the attachment interface 806 to force the tabs 808 thereof into engagement with the retention platform and to align the pipette tip perpendicular to the biasing platform.

FIG. 36 shows the engagement section 404 of the plunger of the pipette tip in engagement with the gripper jaw 184 of the gripper mechanism. During operation of the pipette, the engagement section 404 becomes lockingly gripped by the gripper mechanism of the distal section 145 of the pipette 130 by actuating (e.g., depressing) the lock button disposed in the proximal section 135 of the pipette 130. In embodiments, the second actuator is operable to facilitate advancement of the gripper jaw 184 distally and into engagement with the engagement section 404 of the plunger of a pipette tip that is attached to the pipette via the tip interface 188. During operation of the pipette, the gripper mechanism is actuated by depressing the lock button to lock the engagement section of the plunger within the gripper jaw. Movement of the plunger is subsequently controlled by actuation of the first actuator and the second actuator to perform aspiration and dispensing functions.

In embodiments, the gripper jaw is advanced into engagement with the engagement section 404 simultaneously with advancement of the tabs 808 of the attachment interface 806 of the pipette tip into engagement with the retention platform 955 (e.g., by having the second actuator in the first depressed position, the lock mechanism being in the first configuration (via actuation of the third actuator) while pressing the distal section of the pipette and the attachment interface of the pipette tip into one another). Alternatively, the gripper jaw is advanced into engagement with the engagement section 404 asynchronously with advancement of the tabs into engagement with the retention platform 955 by first pressing the tabs 808 into engagement with the retention platform 955 and subsequently moving the second actuator into the first depressed position. Alternatively, the gripper jaw is advanced into engagement with the engagement section 404 asynchronously with advancement of the tabs into engagement with the retention platform 955 by pressing the tabs 808 into engagement with the retention platform 955 after moving the second actuator into the first depressed position and subsequently moving the first actuator in the first depressed position.

The gripper mechanism includes a gripping sleeve 186 positioned around the gripper jaw 184. The gripping sleeve 186 is configured to exert an inward force on the gripper jaw 184 to cause the gripper jaw 184 to exert an inward force on the engagement section 404 to retain the engagement section of the plunger within the gripper jaw 184.

After gripping and locking (by depressing the lock button) the plunger in engagement with the gripper mechanism, the plunger is controlled by the first and second actuators to perform pipetting functions. Once the plunger is locked by the gripper jaw, a sample may be aspirated into or dispensed from the pipette tip.

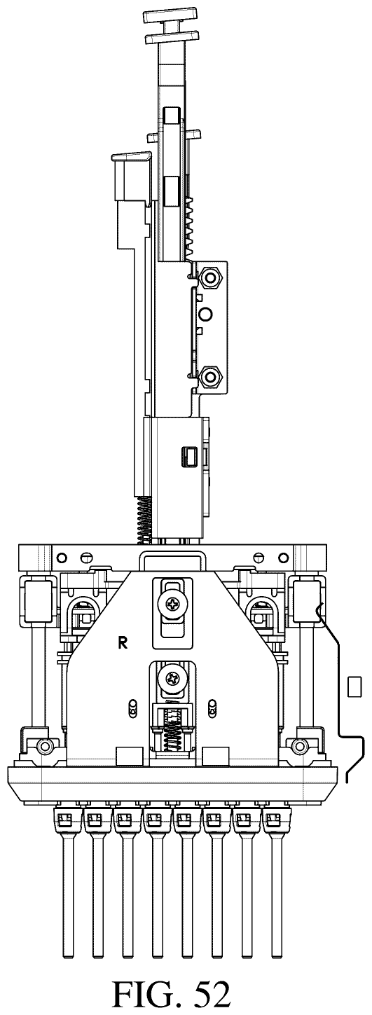

Operation of the pipette to electroporate samples is described as follows in an embodiment of the disclosure. First, a technician grasps the handle of the pipette and holds the pipette with one hand. Next, the second actuator is moved to the first depressed position to move a gripper jaw of each gripper mechanism fully distally and positioned to receive the engagement section of a pipette tip (see FIGS. 25 and 32). This action brings magnets located in the outer housing of the pipette into magnetic alignment with magnets disposed on the linkage bar to hold the first actuator in the first undepressed position (see FIG. 52). Next, the third actuator is actuated by depressing the actuator to unlock the gripper mechanisms which causes the lock mechanism, in operable connection to the third actuator, to transition to the second unlocked configuration. Notably, the gripper mechanisms remain in the first unlocked and open configuration once the third actuator is depressed without additional manual manipulation as a result of the unique design of the gripper mechanisms capable of being held in an open configuration without requiring a technician to continually hold a button in the depressed position.

The technician subsequently moves the pipette over pipette tips, aligns the distal section of the pipette with the pipette tips and presses the gripper jaws and surrounding tip interfaces into attachment interfaces of individual pipette tips. This action causes a clip-on connection to be formed such that the pipette tips become attached to the distal section of the pipette and the engagement section of plungers of the pipette tips are received by the open gripper jaws. The lock mechanism is then transitioned to the second locked configuration by depressing the lock button causing the gripper mechanisms to graspingly engage plungers of pipette tips and lock the plungers in grasping engagement with the pipette.

Liquid samples are then aspirated into the pipette tips by moving the first actuator from the first undepressed position to the second depressed position while the distal tips of the pipette tips are in contact with liquid sample. The pipette is then secured into the pipette docking assembly of the electroporation system described herein such that the liquid samples contained within lumens of the pipette tips are in contact with an electrolytic buffer contained within a reservoir of the docking assembly. The liquid samples are electroporated by delivering to the samples electrical current generated in the pulse generator which is in electrical connection with the electrolytic buffer of the reservoir via a first electrode electrically connected to the pulse generator, and the electrically conductive plunger of each pipette tip via a second electrode contained within the pipette and electrically connected to the pulse generator. Electroporated samples are then dispensed from the pipette tips into a desired receptacle by transitioning the second actuator from the second undepressed position to the first depressed position.

After samples are dispensed, the pipette tips may be disengaged (e.g., ejected) from the pipette by a one, or two-step process. In the one step process, pipette tips are disengaged by actuating the third actuator by fully depressing the actuator. Alternatively, the two-step process may be used to reduce the amount of peak force required from a user to disengage the pipette tips which includes partially depressing the third actuator a first time to separate tabs of each retention interface of attached pipette tips from retention platforms of the pipette while plungers remain graspingly locked within gripper jaws, and subsequently fully depressing the third actuator to transition the lock mechanism from the second locked configuration to the first unlocked configuration (in which the gripper jaws are open) such that the engagement sections of plungers are released from the gripper jaws. In embodiments, a one-step ejection process may require a peak force of about 60 N, and a two-step ejection process as presently disclosed may require a peak force of about 40 N.

As discussed herein, the pipette 130 includes a third actuator that is operable to facilitate disengagement of pipette tips attached to the distal section of the pipette. As shown throughout the Figures, the third actuator 160 is disposed in the proximal section 135 of the pipette 130 and is configured to cause a pipette tip attached to the distal section 145 of the pipette 130 to disengage when the third actuator 160 is actuated (e.g., depressed fully for pipette tip disengagement via the one-step process, or depressed partially and then fully for pipette tip disengagement via the two-step process).

In both processes, partially depressing the third actuator causes a tip ejection sleeve disposed about each tip interface in the distal section of the pipette to move distally with respect to the tip interface and displace the tabs of a pipette tip that are engaged with the retention platform of the tip interface to disengage the attachment interface of the pipette tip from the distal section of the of the pipette (without causing the gripper mechanism to disengage from the plunger). Simultaneously, with disengagement of the attachment interface of the pipette tip, the biasing platform contacting the attachment interface is urged distally via force exerted from compressed springs operably connected to the biasing platform to eject the attachment interface distally.

Further, in both processes, fully depressing the third actuator also causes the lock mechanism to transition from the second locked configuration to the first unlocked configuration thereby transitioning the gripper mechanism to an open configuration such that the engagement section of the plunger of the pipette tip is disengaged and released from the gripper jaw. It will be understood that after the third actuator is fully depressed, the entire pipette tip (e.g., including the plunger and the attachment interface) is disengaged with the pipette.

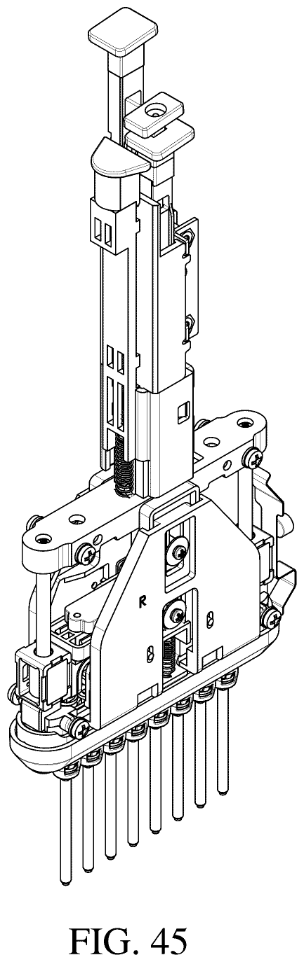

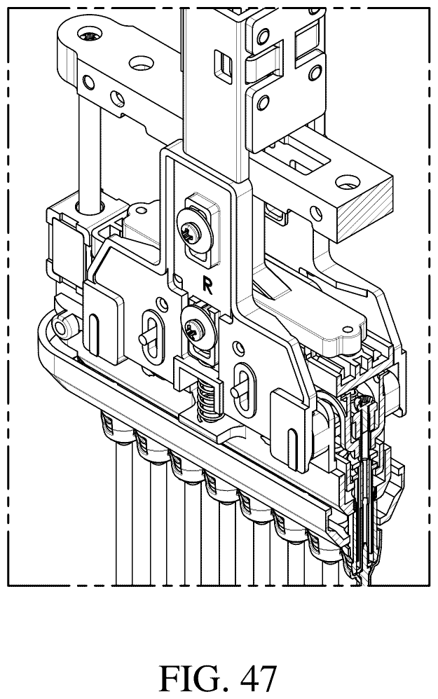

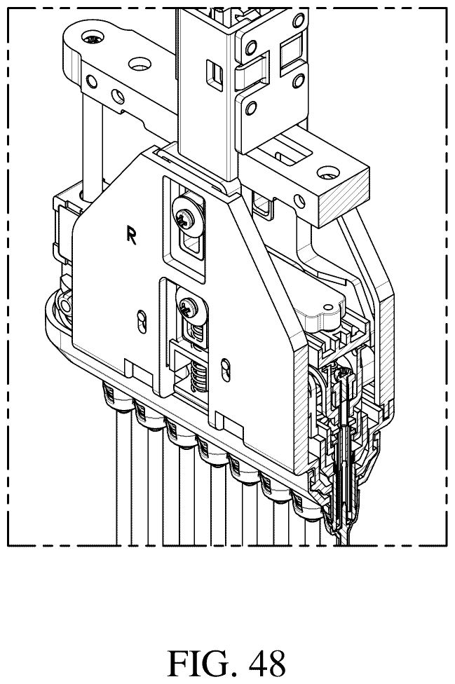

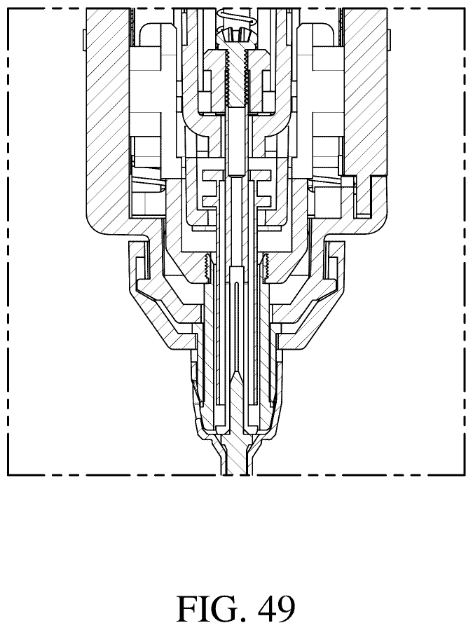

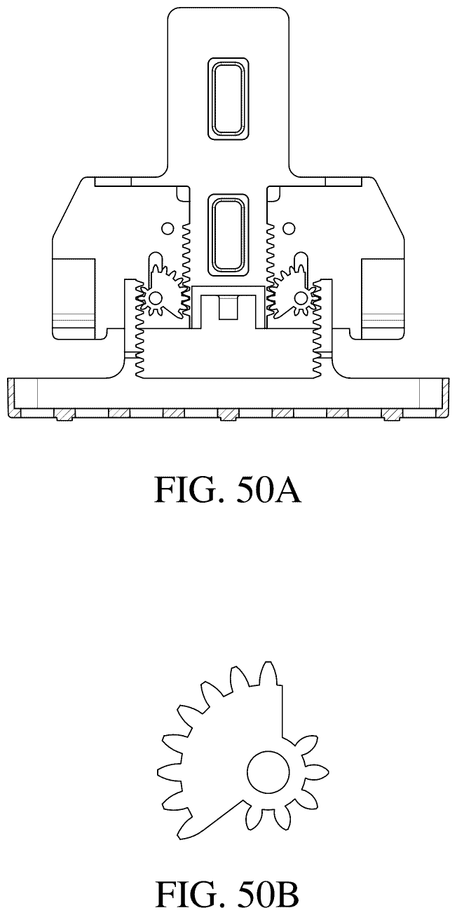

FIGS. 45-49 illustrate pipette tip disengagement (e.g., pipette tip ejection) from the pipette embodiment shown in FIG. 31 using the one-step process. With the second actuator 155 in the first depressed position (e.g., after a dispensing function is performed), fully depressing the third actuator 160 (e.g., eject button) causes the tip ejection sleeve 190 (see FIG. 49) to move distally with respect to the tip interface 188 to disengage the attachment interface via distally oriented force translated through a clip-tip release plate 750, and disengagement of the engagement section of the plunger by movement of the gripping sleeve proximally with respect to the distal end of the gripper jaw caused by distally oriented movement of a plunger release plate 760 resulting in proximal movement of a lifter plate 770 that only occurs when the third actuator is fully depressed. Separation of the fully disengaged pipette tip from the distal section of the pipette is facilitated by distally oriented force exerted on the pipette tip via the biasing platform.

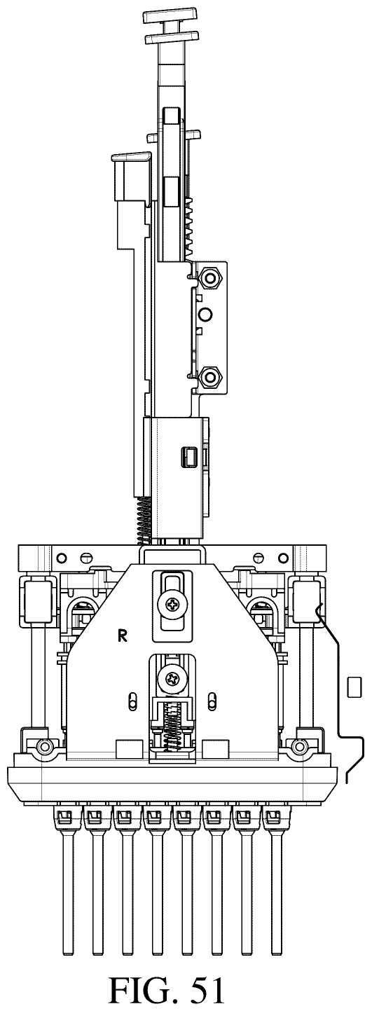

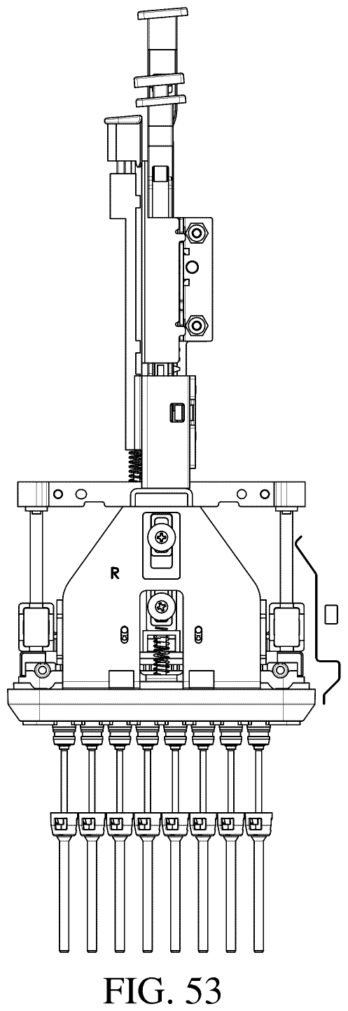

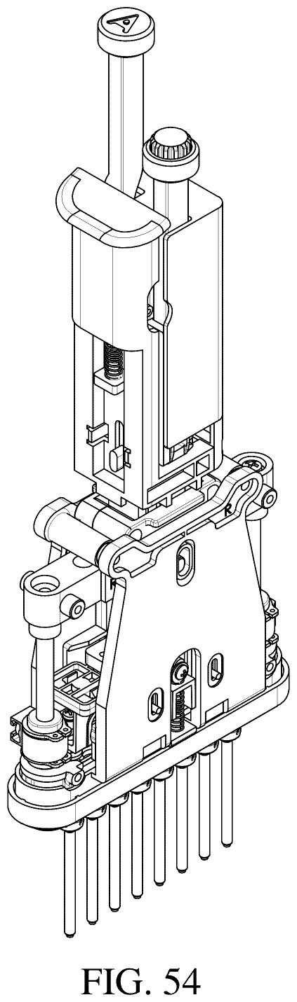

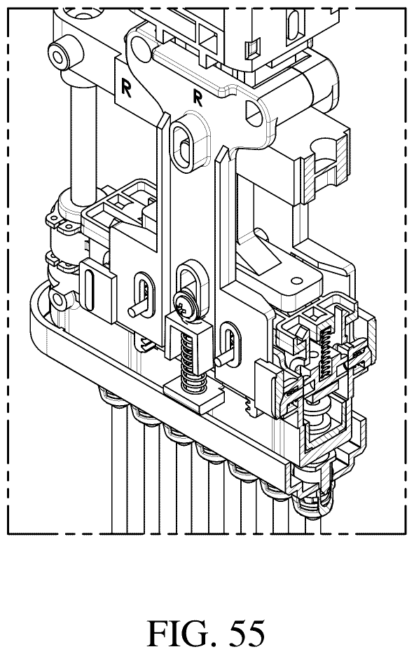

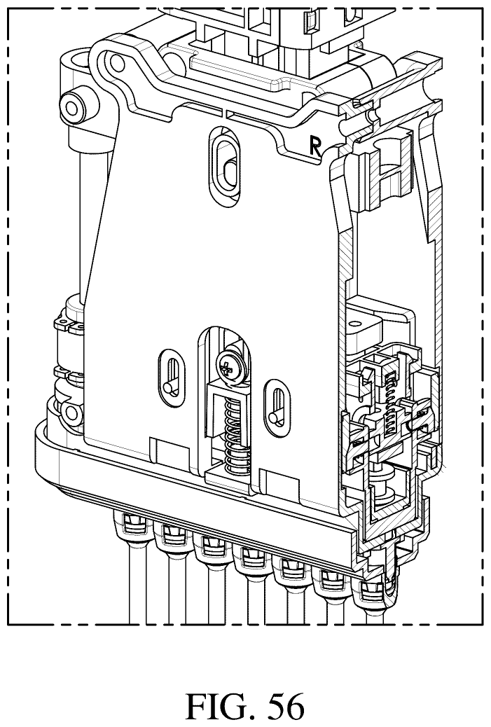

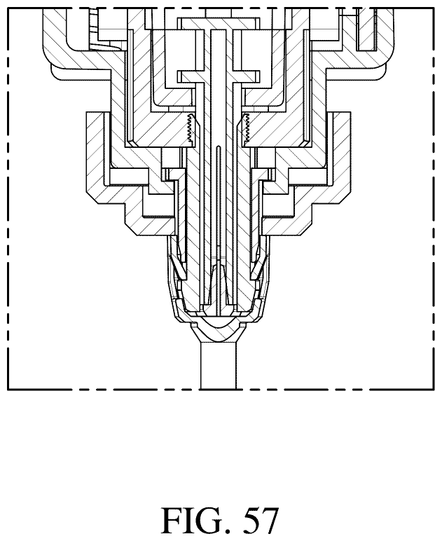

FIGS. 51-53 illustrate pipette tip disengagement (e.g., pipette tip ejection) from the pipette embodiment shown in FIG. 31 using the two-step process. With the second actuator in the first depressed position (e.g., after a dispensing function is performed), the third actuator (e.g., eject button) is partially depressing causing the tip ejection sleeve (see FIG. 49) to move distally with respect to the tip interface to disengage the attachment interface via distally oriented force translated through a release plate. FIG. 53 illustrates partial disengagement of the pipette tip after the third actuator is partially depressed and tip ejection sleeve has dislodged the attachment interface of the pipette tip from the distal section of the pipette. Separation of the attachment interface of the partially disengaged pipette tip from the distal section of the pipette is facilitated by distally oriented force exerted on the pipette tip via the biasing platform. The third actuator is then fully depressed to disengage the engagement section of the plunger by movement of the gripping sleeve proximally with respect to the distal end of the gripper jaw caused by distally oriented movement of a plunger release plate resulting in proximal movement of a lifter plate that only occurs when the third actuator is fully depressed.

As discussed herein, the third actuator 160, when partially or fully depressed, may cause actuation of a tip ejection sleeve 190 to cause the tip ejection sleeve to distally advance toward the tabs 808 (e.g., downward) from an inner side of the tabs 808. During distally oriented advancement, the ejection sleeve 190 may press on the inner side of the tabs 808 to bend the tabs 808 outward and cause the tabs 808 to disengage from the retention platform 955. The biasing member 962 (via contact with biasing platform 964) may then press the attachment interface 806 of the pipette tip 802 downward from the pipette 130, thereby ejecting the attachment interface 806 (and the lumen attached thereto) of the pipette tip 802 from the pipette.

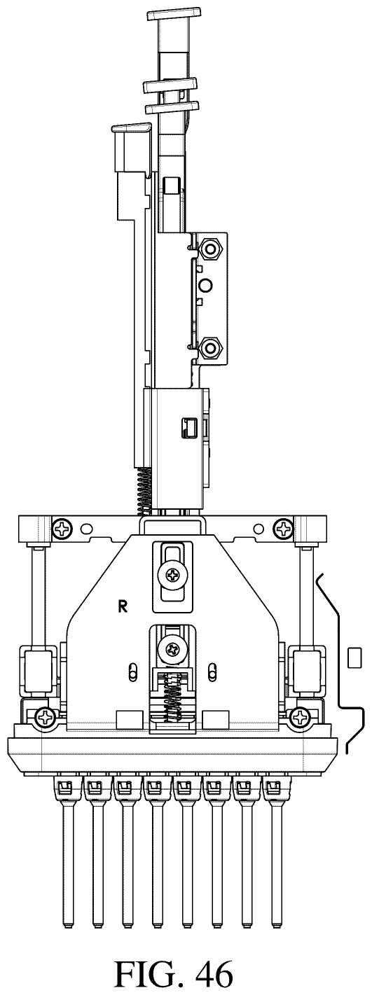

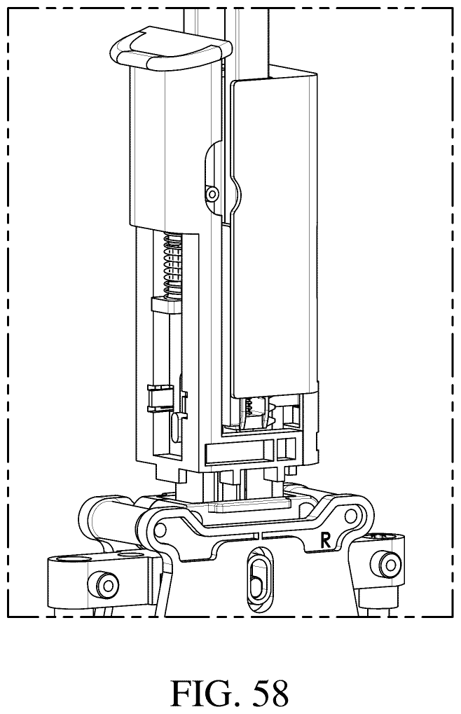

In embodiments, the third actuator 160 is configured to traverse a blank travel distance when pressed (see FIGS. 46 and 58) prior to causing disengagement of the attachment interface 806 and lumen of the pipette tip from the pipette 130 (which occurs by depressing the third actuator 160 through a final distance). Such functionality is designed to prevent inadvertent ejection of the attachment interface and lumen of the pipette tip from the pipette 130.

### Pipette Docking Assembly

In many existing electroporation systems, the reservoir (also referred to herein as “buffer tube”) that holds the electrolytic buffer solution is easily removed from the pipette station (also referred to herein as “pipette docking station”, “pipette station” or “docking station”), which can allow inadvertent removal of the reservoir from the docking station when withdrawing the pipette tip from the reservoir. Such inadvertent removal can result in spillage and/or damage to pipette tips. In some cases, the reservoir (e.g., buffer tube) is held by a pipette station guard (also referred to herein as “station guard”) associated with the docking station (e.g., for protecting users against electrical shock), but conventional pipette station guards can also be inadvertently removed during withdrawal of a pipette from a reservoir (or even during electroporation, which can present an electrical shock hazard).

In embodiments, the present disclosure provides a pipette station guard that locks into the docking station via movement of the station guard in a locking direction that is different from the pipette removal direction for removing the pipette from the reservoir. The reservoir inserts into an opening of the station guard and locks to the station guard. The reservoir can be released from the station guard by application of force (e.g., on latching members) in a force application direction that is the same or different from the pipette removal direction for removing the pipette from the reservoir. Such features may reduce or eliminate the incidence of inadvertent removal of reservoirs (or other reservoirs) and/or station guards from pipette docking stations during pipette removal, thereby reducing or avoiding spillage and/or pipette tip damage.

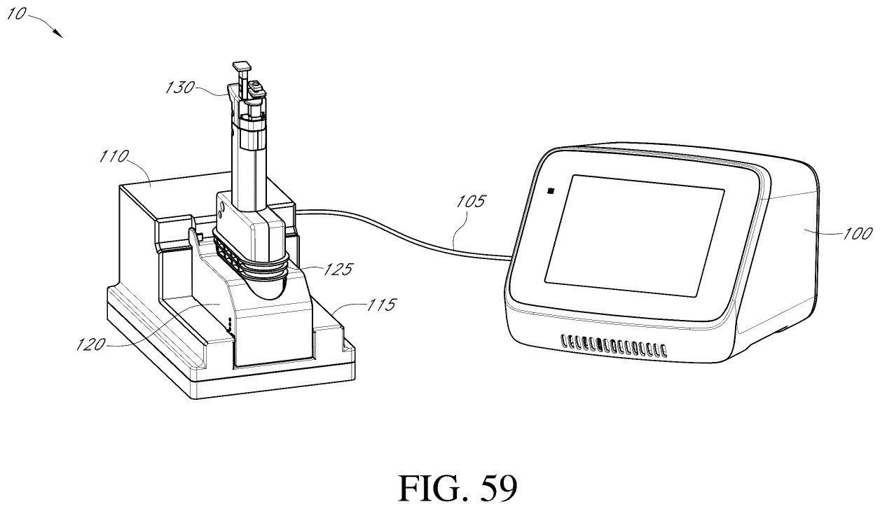

FIG. 59 illustrates an electroporation system 10 of the disclosure including a pipette docking assembly 110 electrically coupled to a pulse generator 100. Also shown in FIG. 59 is a pipette 130 coupled to the pipette docking assembly 110 positioned to perform an electroporation procedure. The distal section of pipette 130 (and attached pipette tips) is docked in the pipette docking assembly 110 such that the pipette is received within the reservoir 125 which is in turn received by the station guard 120.

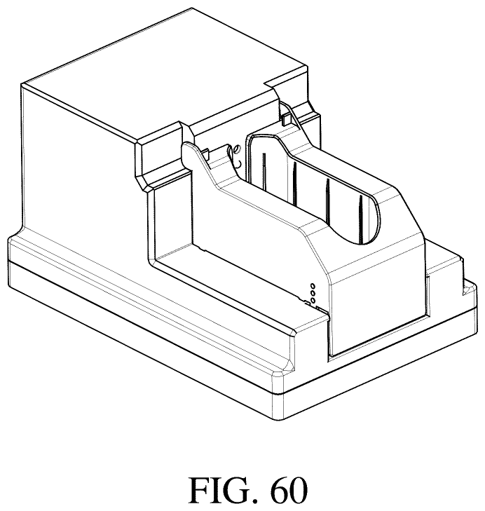

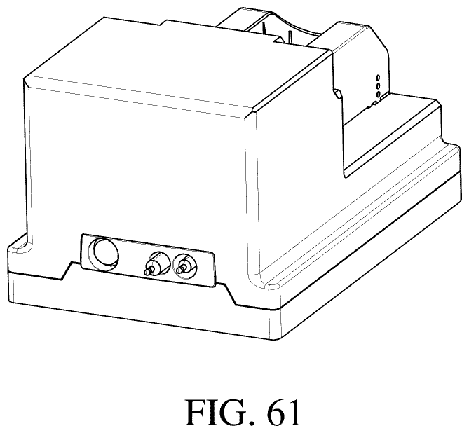

FIGS. 60 and 61 illustrate front and back views of the pipette docking assembly 110 shown in FIG. 59 without the pipette 130 and reservoir 125 being docked.

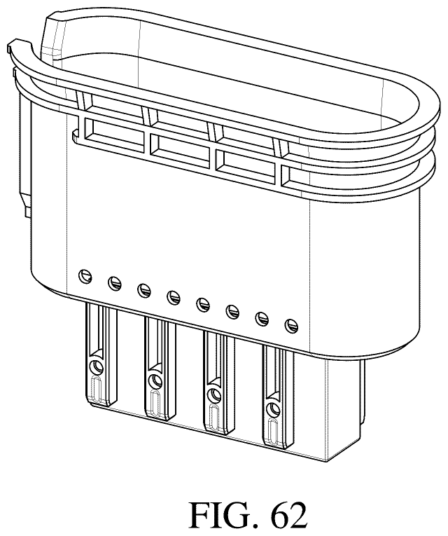

FIG. 62 illustrates aspects of the reservoir 125 used with the pipette docking assembly 110 shown in FIG. 59. In embodiments, the reservoir 125 includes multiple electrodes 117, typically one corresponding to each channel of the pipette. The reservoir shown in FIG. 62 includes 8 electrodes 117 for use with a pipette configured to use 8 pipette tips. It will be understood that the reservoir 125 may include from 2-16 electrodes for use with a pipette configured to use a corresponding number of pipette tips. The electrodes 117 of the reservoir are electrically connected to the pulse generate 100 via electrodes disposed in the docking station 115 when the reservoir 125 is docked in the docking station 115 along with the station guard 120.

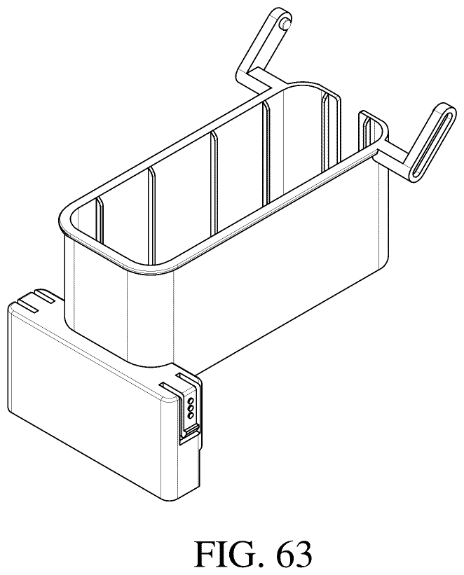

FIG. 63 illustrates a pipette station guard 120 in an embodiment of the disclosure. The pipette station guard 120 may be affixed to a pipette docking station 115 to protect users against potential electrical shock. The pipette station guard 120 may additionally include a reservoir opening for receiving a buffer reservoir (e.g., reservoir 125), which can receive a pipette (e.g., pipette 130) and/or components connected thereto (e.g., a consumable pipette tip).

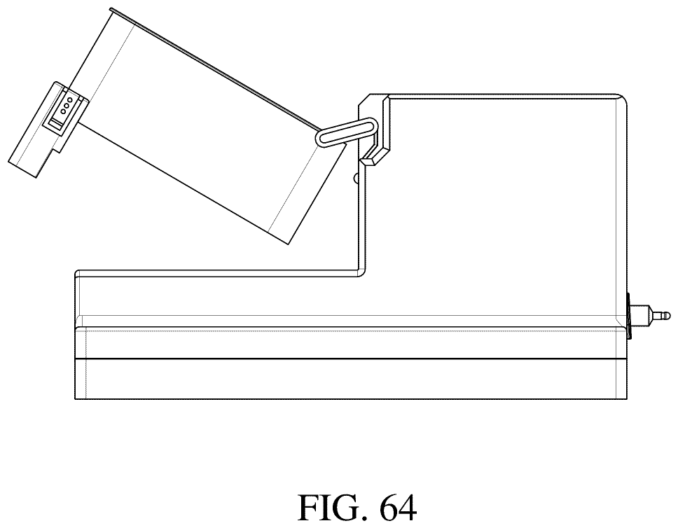

To facilitate connection to a pipette docking station 115, the pipette station guard 120 may include various connection elements, such as one or more locking hooks that are configured to engage with one or more corresponding hook catches of the pipette docking station. Such locking hook(s) may take on various forms. For instance, the example of FIG. 63 illustrates the pipette station guard 120 as including one or more pivot hooks 122, which are configured to rotate into engagement with one or more hook catches (e.g., pivot hook catches) of the pipette docking station (see FIG. 64).

In the example of FIG. 63, the pipette station guard 120 includes a pair of pivot hooks 122 arranged on the rear surface of the pipette station guard 120. The pair of pivot hooks 122 is arranged on the top portion of the rear surface.

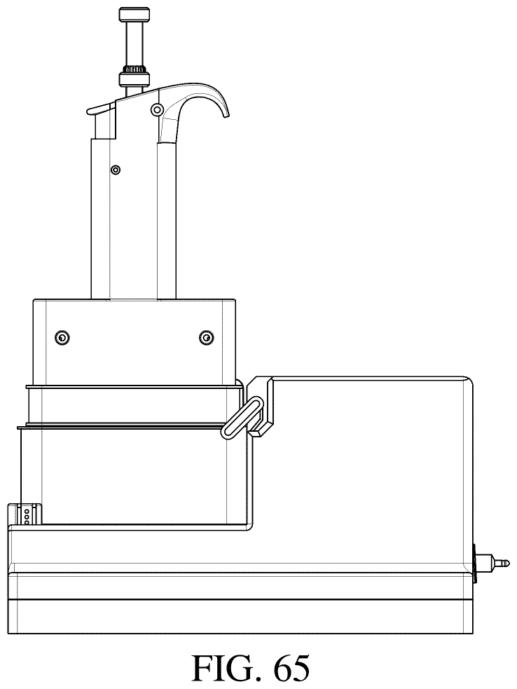

FIG. 64 illustrates the pipette station guard 120 being moved into engagement with a docking station 115. After insertion of the pivot hooks 122 into receiving points of the docking station, the station guard 120 is rotated until an opposing latches 124 reach and interlock with corresponding catches as shown in FIG. 65.

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, processes, and/or products according to certain embodiments of the present disclosure may include, incorporate, or otherwise include properties features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, processes, products, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

Exemplary Subject Matter of the Invention is represented by the following clauses:
Clause 1: A multichannel pipette comprising:
a proximal section having a handle;
a distal section configured to reversibly engage a plurality of pipette tips;
a first actuator disposed in the proximal section; and
a second actuator disposed in the proximal section operable to control a dispensing function of the multichannel pipette,
wherein the first actuator is operable to control an aspiration function and the second actuator is operable to control the dispensing function.
Clause 2: The multichannel pipette of clause 1, further comprising a third actuator disposed in the proximal section configured to cause a pipette tip attached to the distal section of the multichannel pipette to disengage when the third actuator is actuated.
Clause 3: The multichannel pipette of clause 2, wherein the first actuator is configured to transition from a first undepressed position to a second depressed position, wherein the second actuator is configured to transition from a first depressed position to a second undepressed position when the first actuator is transitioned from the first undepressed position to the second depressed position, and wherein transitioning the first actuator from the first undepressed position to the second depressed position causes an aspiration function and transitioning of the second actuator from the second undepressed position to the first depressed position causes a dispensing function.
Clause 4: The multichannel pipette of clause 3, wherein the aspirate function comprises aspiration of fluid into a pipette tip engaged with the distal section of the multichannel pipette.
Clause 5: The multichannel pipette of clause 3, wherein the dispensing function comprises dispensing of fluid from a pipette tip engaged with the distal section of the multichannel pipette.
Clause 6: The multichannel pipette of clause 2, further comprising a plurality of gripper mechanisms disposed in the distal section, each gripper mechanism being configured to reversibly grip a plunger disposed within a lumen of a pipette tip, the plunger having an engagement section for engaging the gripper mechanism and a lumen section disposed within a lumen of a pipette tip.
Clause 7: The multichannel pipette of clause 6, further comprising a lock button disposed in the proximal section operably connected to a lock mechanism configured to control locking of the plurality of gripper mechanisms, wherein depressing the lock button causes the lock mechanism to transition from a first unlocked configuration to a second locked configuration, wherein when the lock mechanism is in the first configuration the plurality of gripper mechanisms are open and configured to receive the engagement section of the plunger, and when the lock mechanism is in the second configuration the plurality of gripper mechanisms are closed and configured to be in grasping engagement with the engagement section of the plunger.
Clause 8: The multichannel pipette of clause 7, wherein the lock mechanism is transitioned from the second locked configuration to the first unlocked configuration when the third actuator is actuated.
Clause 9: The multichannel pipette of clause 7 or clause 8, wherein each of the plurality of gripper mechanisms comprises:
a gripper jaw, the gripper jaw comprising a jaw opening for receiving and retaining the engagement section; and
a gripping sleeve positioned around the gripper jaw and configured to exert an inward force on the gripper jaw to cause the gripper jaw to exert an inward force on the engagement section of the plunger to retain the engagement section of the plunger within the gripper jaw when the lock mechanism is in the second locked configuration.
Clause 10: The multichannel pipette of clause 9, wherein depressing the lock button causes the gripping sleeve to move distally with respect to the gripper jaw to exert the inward force on the gripper jaw
Clause 11: The multichannel pipette of clause 10, wherein actuating the third actuator causes the gripping sleeve to move proximally with respect to the gripper jaw to retract the inward force on the gripper jaw.
Clause 12: The multichannel pipette of clause 11, wherein the first actuator comprises a button disposed at the proximal end of an elongated shaft, and wherein the second actuator comprises a button disposed at the proximal end of an elongated shaft.
Clause 13: The multichannel pipette of clause 12, wherein the lock button is disposed at the distal end of a shaft, and wherein the shaft of the lock button and the shaft of the second actuator are parallel and wherein the lock button and the shaft of the lock button translate with the button of the second actuator and the shaft of the second actuator when the second actuator is moved from the first depressed position to the second undepressed position or from the second undepressed position to the first depressed position.
Clause 14: The multichannel pipette of clause 13, wherein the lock mechanism comprises a push plate in reversible contact with a proximal end of the gripping sleeve, and wherein depressing the lock button causes the push plate to move the gripping sleeve distally with respect to the gripper jaw.
Clause 15: The multichannel pipette of clause 14, wherein actuating the third actuator causes a tip ejection sleeve to move distally with respect to the gripper jaw and contact a proximal end of a pipette tip disposed circumferentially around the gripper jaw to disengage the pipette tip.
Clause 16: The multichannel pipette of clause 15, wherein transitioning the first actuator from the first undepressed position to the second depressed position while the lock mechanism is in the second configuration and engaged with the engagement section of the plunger causes the plunger to move proximally with respect to the gripper jaw thereby aspirating fluid into the pipette tip via a suction force generated by proximal movement of the lumen section within the lumen of the pipette tip.
Clause 17: The multichannel pipette of clause 16, wherein transitioning the second actuator from the second undepressed position to the first depressed position while the lock mechanism is in the second configuration and engaged with the engagement section of the plunger causes the plunger to move distally with respect to the gripper jaw thereby dispensing fluid from the pipette tip via a displacement force generated by distal movement of the lumen section within the lumen of the pipette tip.
Clause 18: The multichannel pipette of clause 6, wherein the multichannel pipette comprises 2, 3, 4, 5, 6, 7, 8 or more gripper mechanisms.
Clause 19: The multichannel pipette of clause 18, wherein the multichannel pipette comprises 8 gripper mechanisms.
Clause 20: The multichannel pipette of any one of the preceding clauses, further comprising a tip interface disposed circumferentially about each gripper jaw, the tip interface including a retention platform configured to engage tabs of the retention interface of a pipette tip to secure the pipette tip to the pipette.
Clause 21: The multichannel pipette of any one of the preceding clauses, further comprising a tip ejection sleeve disposed about the tip interface operable to move distally with respect to the tip interface when the third actuator is actuated and displace the attachment interface of the pipette tip to remove the pipette tip from the pipette.
Clause 22: The multichannel pipette of any one the preceding clauses, further comprising an electrode disposed in the distal section, wherein the electrode is electrically coupled to each gripper jaw of the plurality of gripper mechanisms.
Clause 23: The multichannel pipette of clause 22, wherein each gripper jaw is composed of an electrically conductive material and operable to allow an electrical pulse applied to the electrode to pass through the electrode, through each gripper jaw, through the plunger of the pipette tip, through a sample contained with the lumen of the pipette tip, and through a second electrode disposed adjacent a distal end of the pipette tip, thereby electroporating cells contained within the sample.
Clause 24: An electroporation system comprising:
a multichannel pipette of any one of clauses 1-23;
a pipette tip;
a pipette docking assembly; and
a pulse generator.
Clause 25: The electroporation system of clause 24, wherein the pipette docking assembly comprises a pipette station, a pipette station guard, and a reservoir.
Clause 26: The electroporation system of clause 25, wherein the reservoir comprises a buffer.
Clause 27: The electroporation system of any one of clauses 24-26, wherein the pipette tip comprises a 10 μL pipette tip or a 100 μL pipette tip.
Clause 28: The electroporation system of any one of clauses 24-27, wherein the pipette tip comprises a plunger at least partially disposed within a lumen, the plunger being configured to translate along the lumen to facilitate aspirating and/or dispensing.
Clause 29: The electroporation system of clause 28, wherein the plunger comprises gold, diamond-like carbon, and/or conductive plastic.
Clause 30: The electroporation system of clause 28 or clause 29, wherein the plunger comprises an engagement section and a lumen section, the lumen section comprising a sealing component for creating a seal between the plunger and the lumen.
Clause 31: The electroporation system of clause 30, wherein the lumen section comprises a front pin and a shaft section, wherein the front pin is configured to connect to the shaft section and secure the sealing component to shaft section.
Clause 32: The electroporation system of clause 31, wherein the sealing component comprises a polymer sleeve, and wherein the front pin is configured to insert through the polymer sleeve and engage with a retention hole of the shaft section to secure the sealing component to the shaft section.
Clause 33: The electroporation system of clause 32, wherein insertion of the front pin through the polymer sleeve defines a space between the polymer sleeve and the front pin, and wherein the space contributes to a flexibility of the polymer sleeve for creating the seal between the plunger and the lumen.
Clause 34: The electroporation system of any one of clauses 30-33, wherein the sealing component comprises polytetrafluoroethylene (PTFE).
Clause 35: The electroporation system of clause 30, wherein the sealing component comprises a coated O-ring, and wherein the lumen section comprises a circumferential depression configured to receive the coated O-ring.
Clause 36: The electroporation system of any one of clauses 30-35, wherein the pipette tip further comprises an attachment interface adjacent to the lumen, the attachment interface comprising one or more tabs configured to engage with a retention platform of a distal section of the pipette assembly.
Clause 37: The electroporation system of any one of clauses 24-36, wherein:
the pipette tip is connected to the multichannel pipette and positioned within the reservoir and in contact with the buffer,
a first electrode is electrically coupled with the buffer,
the plunger of the pipette tip is in electrical communication with a sample within the pipette tip and a pipette electrode, and
the pipette electrode is in contact with a second electrode, the first and second electrodes being in electrical communication with the pulse generator.
Clause 38: The electroporation system of any one of clauses 24-37, wherein the pulse generator comprises one or more voltage sources configured to charge one or more high-voltage capacitors, the one or more high-voltage capacitors being configured to operate as a power supply for an amplifier circuit, the amplifier circuit being configured to supply voltage to a sample of the multichannel pipette in a manner that accounts for variations in load.
Clause 39: The electroporation system of clause 38, wherein the amplifier circuit comprises a common source amplifier configured to output a high-voltage pulse.
Clause 40: The electroporation system of clause 39, wherein the common source amplifier receives a signal from an amplitude setting loop, wherein the signal of the amplitude setting loop is based upon input from a digital-to-analog converter and input from a voltage sensing loop, wherein the input from the voltage sensing loop is determined using the high-voltage pulse, a voltage divider, and a differential amplifier.
Clause 41: The electroporation system of clause 40, wherein the common source amplifier amplifies the signal from the amplitude setting loop by a factor of about 1,000 to about 2,000.
Clause 42: The electroporation system of clause 40 or clause 41, wherein the input from the digital-to-analog converter corresponds to a user-selected waveform.
Clause 43: The electroporation system of any one of clauses 24-42, wherein the pulse generator comprises an arcing detection module configured to detect arcing within a sample during application of voltage to the sample.
Clause 44: The electroporation system of clause 43, wherein the arcing detection module comprises:
a first stage amplifier configured to provide an amplified current signal based on a current signal associated with the application of the voltage to the sample;
a bandpass filter configured to filter a falling edge signal from the amplified current signal, the falling edge signal being indicative of a drop in current passing through the sample, the drop in current being suggestive of arcing, and
a comparator configured to compare the falling edge signal filtered by the bandpass filter to one or more reference criteria to determine whether arcing has occurred in the sample.
Clause 45: The electroporation system of clause 44, wherein amplification of the current signal by the first stage amplifier is based upon output of low voltage detection circuitry for determining resistance associated with the sample.
Clause 46: A method of transfecting a cell with a payload, comprising:
providing an electroporation system of any one of clauses 24 through 45;
providing the cell;
providing the payload;
introducing the cell and the payload into the pipette tip; and
electroporating the cell by operating the electroporation system.
Clause 47: The method of clause 46, wherein the cell is a mammalian cell.
Clause 48: The method of clause 46, wherein the cell is a microbe or organoid.
Clause 49: The method of clause 46, wherein the payload is selected from the group consisting of a nucleic acid, a protein, or a combination thereof.

1. A multichannel pipette comprising:
a proximal section having a handle;
a distal section configured to reversibly engage a plurality of pipette tips;
a first actuator disposed in the proximal section; and
a second actuator disposed in the proximal section operable to control a dispensing function of the multichannel pipette,
wherein the first actuator is operable to control an aspiration function and the second actuator is operable to control the dispensing function.

2. The multichannel pipette of claim 1, further comprising a third actuator disposed in the proximal section configured to cause a pipette tip attached to the distal section of the multichannel pipette to disengage when the third actuator is actuated.

3. The multichannel pipette of claim 2, wherein the first actuator is configured to transition from a first undepressed position to a second depressed position, wherein the second actuator is configured to transition from a first depressed position to a second undepressed position when the first actuator is transitioned from the first undepressed position to the second depressed position, and wherein transitioning the first actuator from the first undepressed position to the second depressed position causes an aspiration function and transitioning of the second actuator from the second undepressed position to the first depressed position causes a dispensing function.

4. The multichannel pipette of claim 3, wherein the aspirate function comprises aspiration of fluid into a pipette tip engaged with the distal section of the multichannel pipette.

5. The multichannel pipette of claim 3, wherein the dispensing function comprises dispensing of fluid from a pipette tip engaged with the distal section of the multichannel pipette.

6. The multichannel pipette of claim 2, further comprising a plurality of gripper mechanisms disposed in the distal section, each gripper mechanism being configured to reversibly grip a plunger disposed within a lumen of a pipette tip, the plunger having an engagement section for engaging the gripper mechanism and a lumen section disposed within a lumen of a pipette tip.

7. The multichannel pipette of claim 6, further comprising a lock button disposed in the proximal section operably connected to a lock mechanism configured to control locking of the plurality of gripper mechanisms, wherein depressing the lock button causes the lock mechanism to transition from a first unlocked configuration to a second locked configuration, wherein when the lock mechanism is in the first configuration the plurality of gripper mechanisms are open and configured to receive the engagement section of the plunger, and when the lock mechanism is in the second configuration the plurality of gripper mechanisms are closed and configured to be in grasping engagement with the engagement section of the plunger.

8. (canceled)

9. The multichannel pipette of claim 7, wherein each of the plurality of gripper mechanisms comprises:
a gripper jaw, the gripper jaw comprising a jaw opening for receiving and retaining the engagement section; and
a gripping sleeve positioned around the gripper jaw and configured to exert an inward force on the gripper jaw to cause the gripper jaw to exert an inward force on the engagement section of the plunger to retain the engagement section of the plunger within the gripper jaw when the lock mechanism is in the second locked configuration.

10. The multichannel pipette of claim 9, wherein depressing the lock button causes the gripping sleeve to move distally with respect to the gripper jaw to exert the inward force on the gripper jaw.

11. The multichannel pipette of claim 10, wherein actuating the third actuator causes the gripping sleeve to move proximally with respect to the gripper jaw to retract the inward force on the gripper jaw.

12. The multichannel pipette of claim 11, wherein the first actuator comprises a button disposed at the proximal end of an elongated shaft, and wherein the second actuator comprises a button disposed at the proximal end of an elongated shaft.

13. The multichannel pipette of claim 12, wherein the lock button is disposed at the distal end of a shaft, and wherein the shaft of the lock button and the shaft of the second actuator are parallel and wherein the lock button and the shaft of the lock button translate with the button of the second actuator and the shaft of the second actuator when the second actuator is moved from the first depressed position to the second undepressed position or from the second undepressed position to the first depressed position.

14. The multichannel pipette of claim 13, wherein the lock mechanism comprises a push plate in reversible contact with a proximal end of the gripping sleeve, and wherein depressing the lock button causes the push plate to move the gripping sleeve distally with respect to the gripper jaw.

15. The multichannel pipette of claim 14, wherein actuating the third actuator causes a tip ejection sleeve to move distally with respect to the gripper jaw and contact a proximal end of a pipette tip disposed circumferentially around the gripper jaw to disengage the pipette tip.

16. The multichannel pipette of claim 15, wherein transitioning the first actuator from the first undepressed position to the second depressed position while the lock mechanism is in the second configuration and engaged with the engagement section of the plunger causes the plunger to move proximally with respect to the gripper jaw thereby aspirating fluid into the pipette tip via a suction force generated by proximal movement of the lumen section within the lumen of the pipette tip.

17. The multichannel pipette of claim 16, wherein transitioning the second actuator from the second undepressed position to the first depressed position while the lock mechanism is in the second configuration and engaged with the engagement section of the plunger causes the plunger to move distally with respect to the gripper jaw thereby dispensing fluid from the pipette tip via a displacement force generated by distal movement of the lumen section within the lumen of the pipette tip.

18. The multichannel pipette of claim 6, wherein the multichannel pipette comprises 2, 3, 4, 5, 6, 7, 8 or more gripper mechanisms.

19-21. (canceled)

22. The multichannel pipette of claim 9, further comprising an electrode disposed in the distal section, wherein the electrode is electrically coupled to each gripper jaw of the plurality of gripper mechanisms.

23. The multichannel pipette of claim 22, wherein each gripper jaw is composed of an electrically conductive material and operable to allow an electrical pulse applied to the electrode to pass through the electrode, through each gripper jaw, through the plunger of the pipette tip, through a sample contained with the lumen of the pipette tip, and through a second electrode disposed adjacent a distal end of the pipette tip, thereby electroporating cells contained within the sample.

24-49. (canceled)
